(19) United States
(12) Patent Application Publication
(10) Pub. No.: US 2007/0010433 A1
(43) Pub. Date: Jan. 11, 2007

(54) Use of compounds capable of inhibiting the proteolytic processing of semaphorins for prevention, treatment, diagnosis and prognosis of an invasive disease

(75) Inventors: Morten Albrechtsen, Chariottenlund (DK); Claus Christensen, Copenhagen (DK); Eugene Lukanidin, Copenhagen (DK); Ole Olsen, Charlottenlund (DK)

Correspondence Address: BROWDY AND NEIMARK, P.L.L.C.;624 NINTH STREET, NW, SUITE 300, WASHINGTON, DC, 20001-5303, US

(73) Assignee: SEMA APS, Charlottenlund (DK)

(21) Appl. No.: 10/520,741

(22) PCT Filed: Jul. 10, 2003
(86) PCT No.: PCT/DK03/00486

§ 371 Date: Jan. 11, 2005

(30) Foreign Application Priority Data
Jul. 11, 2002 (DK) PA 2002 01092

(51) Int. Cl. A61K 38/17

(52) U.S. Cl. 514/12

(57) ABSTRACT

The present invention relates to use of compounds directed to inhibiting expression and/or proteolytic processing semaphorins SEMA3E and/or sema3E and/or activation of a receptor by a proteolytic product of said semaphorins for the manufacture of a medicament for prevention, treatment diagnosis and/or prognosis of an invasive disease. The invention features the compounds selected from the group comprising antisense compounds derived from the sequence of SEMA3E and/or sema3E, peptide compounds derived from the sequence of said semaphorins, anti-bodies against said semaphorins, and peptide compounds derived from the sequence of Plexin A receptor. Furthermore, the invention provides methods for prognosis and/diagnosis of malignancy of cancer based of estimation of the levels of expression and proteolytic processing of said semaphorins in a sample of a tissue or body fluid. The invention also concerns a method for producing an attractant polypeptide by establishing a cleavage product of SEMA3E. and/or sema3E.

### FIELD OF INVENTION

The present invention relates to use of compounds directed to inhibiting expression, and/or proteolytic processing of semaphorins SEMA3E and/or sema3E, and/or activation of the receptor by a proteolytic product of said semaphorins for the manufacture of a medicament for prevention, treatment, diagnosis and/or prognosis of an invasive disease. The invention features the compounds selected from the group comprising antisense compounds derived from the sequence of SEMA3E and/or sema3E, peptide compounds derived from the sequence of said semaphorins, antibodies against said semaphorins, peptide compounds derived from the sequence of Plexin A receptor. Furthermore, the invention provides methods for prognosis and/diagnosis of malignancy of cancer based of estimation of the levels of expression and proteolytic processing of semaphorins in a sample of tissue or body fluid. The invention also concerns a method for producing an attractant polypeptide by establishing a cleavage product of SEMA3E and/or sema3E.

### BACKGROUND OF INVENTION

Malignant tumors shed cells which migrate to new tissues and create secondary tumors; a benign tumor does not generate secondary tumors. The process of generating secondary tumors is called metastasis and is a complex process in which tumor cells colonise sites distant from the primary tumor.

Tumor metastasis remains the major cause of morbidity and death for patients with cancer. One of the greatest challenges in cancer research is to understand the basis of metastasis, i. e., what controls the spread of tumor cells through the blood and lymphatic systems and what allows tumor cells to populate and flourish in new locations.

The metastatic process appears to be sequential and selective, and is controlled by a series of steps since metastatic tumor cells: (a) are mobile and can disseminate from the original tumor; (b) are capable of invading the cellular matrix and penetrating through blood vessels; (c) possess immunological markers, which allow them to survive passage through the blood stream, where they must avoid the immunologically active cytotoxic “T” lymphocytes; and (d) have the ability to find a favourable location to transplant themselves and successfully survive and grow.

Intercellular communication between host and tumor cells is crucial to the remodelling of tissues that permits the growth and metastasis of cancers. Tissue remodelling is a process that strongly depends upon a tight regulation of local expression and activation of different functional groups of proteins. Growing evidence demonstrates a correlation between malignancy of a tumor and deregulated expression of enzymes, that degrade the extracellular matrix, guidance molecules and their receptors, which serve to induce angiogenesis, and proteases, that recognize and cleave precursors of these enzymes and guidance molecules thereby activating them (Bassi et al., 2001, Mol Carcinog 31:224-32). Thus, up-regulated expression of one groups of guidance molecules, semaphorins, which play an important role in development, in particular nervous system, has been recently demonstrated in malignant tumor cells (Christensen et al., 1998, Cancer Res 58:1238-44).

Semaphorins constitute a large family of secreted and membrane bound molecules that are characterised by an extracellular ˜500 amino acid semaphorin domain. Semaphorins were originally characterised in nervous system, where they have been implicated in repulsive axon guidance (Kolodkin et al. 1993, Cell 75:1389-99). More recently, semaphorins have been further implicated in the immune response (Hall et al., 1996, Proc Natl Acad Sci USA 93:11780-5) and regulation of angiogenesis (Miao et al., 2000, FASEB J 14:2532-9). Member of one class of semaphorins, the class 3 secreted semaphorins (Sema3), have been shown to interact with transmembrane molecules called neuropilins (NP-1 and NP-2) (Chen et al., 1997, Neuron 19:547-59). Semaphorins also have other functional receptors called Plexins (Tamagnone et al., 1999, Cell 99:71-80). Both the semaphorins and Plexins have been demonstrated being expressed in tumor cells (Christensen et al., 1998, Cancer Res 58:1238-44; Brambilla et al., 2000, Am J Pathol 56:939-50; Trusolino and Comoglio, 2002, Nature Rev Cancer 2:289-300).

The functions that semaphorins execute in different cells and tissues are contradictory. Interestingly, the role they play in different cells and tissues can sometimes be reversed by mechanisms, which are poorly understood to date. For example, mouse Sema3E being a repulsive cue for sensory and sympathetic neurons inducing the growth cone collapse, (Chen et al., 1997, Neuron 19:547-59; Miyazaki et al., 1999, Neuroscience 93:401-8), in contrary, functions as a chemoattractant and neurite outgrowth inducer for PC12 cells (Sakai et al., 1999, J Biol Chem 274:29666-71). Human SEMA3B and SEMA3F have been proposed to act as tumor suppressors (Xiang et al., 1996, Genomics 32:39-48; Sekido et al., 1996, Proc Nat Acad Sci USA 93:4120-5; Brambilla et al., 2000, Am J Pathol 56:939-50; Tomizawa et al., 2001, Proc Natl Acad Sci USA 98:13954-9). Unlike SEMA3B and SEMA3F, SEMA3C and SEMA3E are thought to promote tumor progression (Martin-Satue and Bblanco 1999, J Surg Oncol 72:18-23; Yamada et al. 1997, Proc Natl Acad Sci USA 94:14713-8; Christensen et al., 1998, Cancer Res 58:1238-44; Williamson et al., 2001, Proceedings of the AACR, Vol. 42. Abs). These contradictory roles the semaphorins play may partially be explained by a suggestion that they bind to and activate different receptors expressed on the surface of different cells, or other mechanisms, such as, for example, extracellular proteolytic processing, may influence their function. It has been shown that the chemorepulsive activity of secreted semaphorins is regulated by furin-dependent proteolytic processing (Adams et al., 1997, EMBO J 16:6077-86).

Furin is a pro-protein convertase that belongs to the group of Ca2+-dependent serine proteases cleaving at paired basic residues. Pro-protein convertases (PCs) are proteases that recognise and cleave precursor proteins, e. g. hormones, growth factors, receptors, etc. PCs play a significant role in tissue remodelling as during development, as in tumor progression. Activity of furin and other PCs has been demonstrated to correlate with the invasive and metastatic potential of tumor cells (Bassi et al., 2001, Proc Natl Acad Sci USA 98:10326-31; Bassi et al., 2001, Mol Carcinog 31:224-32).

Secreted Sema3E is known as a repulsive guidance molecule in developing nervous system (Miyazaki et al., 1999, Neuroscience 93:401-8; Pozas et al., 2001, Mol Cell Neurosci 18:26-43; Castellani et al., 2000, Neuron 27:237-49). It has recently been shown that Sema3E is also expressed in some tumor cells in vivo and in vitro (Christensen et al., 1998, Cancer Res 58:1238-44; Williamson et al., 2001, Proceedings of the AACR vol. 42. Abs). Expression of Sema3E in tumor cells has been correlated with their metastatic potential. However, the role of this protein in malignant tumor cells and the mechanism, which underlies the malignant transformation of tumor cells upon the expression of Sema3E, has not been defined.

### SUMMARY OF THE INVENTION

The present invention relates to the surprising findings that the truncated form of a polypeptide of the semaphorin family derived from proteolytic precessing of said semaphorin influences cell motility and is an attractant for the lymph node and lung microcapillary endotelial cells.

It is an object of the present invention to use (i) an agent capable of inhibiting expression of a polypeptide belonging to the semaphorin family of proteins, and/or (ii) an agent capable of inhibiting intracellular or extracellular proteolytic processing of a polypeptide belonging to the semaphorin family of proteins, wherein the agent is selected from antibodies or fragments of antibodies directed to said polypeptide, or fragments or variants of fragments of said polypeptide, and/or (iii) an agent capable of inhibiting binding a proteolytic fragment of a polypeptide belonging to the semaphorin family of proteins to a receptor and thereby inhibiting sequential activation of said receptor, for the manufacture of a medicament for prevention of progression of an invasive disease in an individual, wherein invasion of cells, other organisms, or invasion of itself plays a role in disease pathogenesis, said invasive disease being selected from the group comprising autoimmune, infectious growth disturbance, neurological, cardiovascular, respiratory, kidney or neoplastic diseases, with the proviso that the disease is not cancer.

Another object of the invention is to use (i) an agent capable of inhibiting expression of a polypeptide belonging to the semaphorin family of proteins, and (ii) an agent capable of inhibiting intracellular or extracellular proteolytic processing of a polypeptide belonging to the semaphorin family of proteins, wherein the agent is selected from antibodies or fragments of antibodies directed to said polypeptide, or fragments or variants of fragments of said polypeptide, and/or (iii) an agent capable of inhibiting binding a proteolytic fragment of a polypeptide belonging to the semaphorin family of proteins to a receptor and thereby inhibiting sequential activation of said receptor, for the manufacture of a medicament for prevention of prevention of metastasis of cancer in vivo and tumor progression in vitro or in vivo.

It is another object of the invention to use (i) an agent capable of inhibiting expression of a polypeptide belonging to the semaphorin family of proteins, and (ii) an agent capable of inhibiting intracellular or extracellular proteolytic processing of a polypeptide belonging to the semaphorin family of proteins, wherein the agent is selected from antibodies or fragments of antibodies directed to said polypeptide, or fragments or variants of fragments of said polypeptide, and/or (iii) an agent capable of inhibiting binding a proteolytic fragment of a polypeptide belonging to the semaphorin family of proteins to a receptor and thereby inhibiting sequential activation of said receptor, for the manufacture of a medicament for prevention of progression of metastasis of cancer in vivo and tumor progression in vitro and/or in vivo. and/or for treatment of malignant forms of cancer.

It is yet another objection of the invention to provide a method for diagnosis of malignant cancer, comprising (i) assessing the level of expression of a semaphorin of the invention in a tumor, and (ii) detecting fragments of the semaphorin of the invention in a body liquid, such as blood, urea or faeces, and (iii) measuring the ratio between a full-length semaphorin of the invention and peptide fragments of said semaphorin in a tumor and/or a body liquid, such as blood, urea or faeces, and a method for prognosis of malignancy of cancer, comprising (i) assessing the level of expression of the semaphorin of the invention in a tumor, (ii) detecting fragments of the semaphorin of the invention in a body liquid, such as blood, urea or faeces, and (iii) measuring the ratio between a full length semaphorin of the invention and peptide fragments of said semaphorin in a tumor and/or a body liquid, such as blood, urea or faeces.

Another important aspect of the invention relates to providing the compounds which are used in the methods described above. In this aspect the invention provides
(i) an antisense compound at about 19 nucleobases in length comprising at least an 5-nucleobase portion of the sequence set forth in SEQ ID NO: 7 or SEQ ID NO: 8, which inhibits expression of semaphorin polypeptides of the invention;
(ii) a peptide fragment capable of binding a proprotein convertase and thereby inhibiting the activity of said convertase;
   (iii) an isolated polyclonal antibody, natural or artificial variants, or antibody fragments, which specifically binds to an epitope located within a sequence of about 9 to about 50 amino acids in length located the structural domain of a semaphorin polypeptide of the invention comprising a proprotein convertase cleavage site RXK/RR, and thereby inhibits the cleavage of said polypeptide on said cleavage site;
(iv) an isolated monoclonal antibody, natural or artificial variants, or peptide fragments of thereof, which specifically binds to an epitope located within a sequence of about 9 to about 50 amino acids in length located in the structural domain of a semaphorin of the invention comprising a proprotein convertase cleavage site RXK/RR, and thereby inhibits the cleavage of said polypeptide on said cleavage site,
(v) a peptide fragment derived from the sequence of a semaphorin of the invention, or variants thereof, capable of binding of a receptor of the Plexin family receptors without activating said receptor;
(vi) a peptide fragment derived from the sequence of ectodomain of a receptor of the Plexin family receptors, or natural or synthetic variants thereof, capable of binding a polypeptide derived from proteolytic cleavage of a semaphorin of the invention by a proprotein convertase.

The invention also provides a method for producing an antibody raised against a semaphorin of the invention, or natural or artificial variants, or peptide fragments thereof, which specifically binds to an epitope located within a sequence of about 9 to about 50 amino acids in length located the structural domain of said semaphorin comprising a proprotein convertase cleavage site RXK/RR, and thereby inhibits the cleavage of said protein on said cleavage site and a hybridoma cell line capable of producing a monoclonal antibody directed to the epitope located within said structural domain.

Moreover the invention relates to the use of the provided compounds described above for the manufacture of a medicament for prevention and/or treatment of metastasis of cancer in vivo or tumor progression in vivo and in vitro, and the use of said compounds for the manufacture of a kit for diagnosis and/or prognosis of malignancy of a cancer.

In an additional aspect the invention concerns using an agent capable of inhibiting proteolytic cleavage a semaphoring, said agent comprisng a compound described above and/or commercially available inhibitors of serine proteases and/or proprotein convertases for the inhibiting activation of a receptor of the Plexin A subfamily, comprising Plexin A1, Plexin A2 and Plexin A3 in vivo.

In another additional aspect the invention relates to a method for producing an attractant polypeptide by establishing a cleavage product or a variant of a cleavage product from a repulsive polypeptide, said repulsive polypeptide being a semaphorin of the invention.

### DESCRIPTION OF DRAWINGS

FIG. 1 shows Northern hybridisations of subclass 3 semaphorins (sema3A, B, C, E and F) to RNA from mouse mammary tumor cell lines and immortalized fibroblasts.

FIG. 2 demonstrates the presence of different isoforms of Sema3E in the medium collected from mock-transfected 67NR, 168FARN and COS-7 cells or the cells transfected with full-length Sema3E, and malignant tumor CSML1000B and 66cl4 cells.

FIG. 3 demonstrates that chromatographic fractions of media collected from COS-7 cells expressing full-length Sema3E that contain p87:p87-Sema3eE dimer (#5 and#6) (C) are capable to induce neurite outgrowth from PC12E2 cells (B) and failes to stimulate motility of SVEC4-10 cells, whereas fractions (#13 and #14) containing the truncated p61-Sema3E isoform are capable to do both the induction of nurites and stimulation of motility.

FIG. 4 shows that the presence of recombinant p61 isoform of Sema3E in the growth medium has is a stimulatory effect on neurite outgrowth from PC12E2 cells.

FIG. 5 demonstrates that the medium collected from transfected 168FARN and COS-7 cells containing secreted Sema3E has a stimulatory effect on motility of SVEC4-10 cells.

FIG. 6 demonstrates a selective stimulatory effect of secreted Sema3E on motility of endothelial cells: the effect is significant only for LE-1 cells, but not for HSE cells and that immunoprecipitation of Sema3E from the medium removes the stimulation of cell motility.

FIG. 7 shows that the p61 isoform of Sema3E recombinantly produced in yeast, is capable of stimulating cell motility (controls: hepatic growth factor (HGF), basic fibroblast growth factor (bFGF), bovine serum albumine (BSA)).

FIG. 8 demonstrates that when A—heparan sulphate proteolycan (HSPG), Perlican, used as a substrate for SVEC4-10 cells, the response of cells to the presence of Sema3e in the medium is a double increase in motility, if compared to cells grown on the substrate without HSPG, and B—addition of heparin to the medium diminishes the stimulatory effect of both HSPG and Sema3E.

FIG. 9 shows that the clones of 168FARN cells expressing the full-length Sema3E (#14 and #2s) are capable to colonise the lungs in the experimental metastasis assay.

FIG. 10 demonstrates that the expression of p61-Sema3E in non-metastatic 168FARN cells is sufficient to bestow the cells a capacity to colonise the lungs in the experimental metastasis assay (negative controls:168FARN cells expressing the full-length Sema3E mutated on the furin cleavage site (Sema3E(−)(+)myc), 168FARN cells expressing alkaline phosphotase (AP); positive control: 168FARN cells expressing the full-length Sema3E fused with AP (AP-Sema3E)).

FIG. 11 shows the p61 isoform of Sema3E secreted by tumor cells in the medium as an attractant for endothelial cells grown in 3D co-culture (keys to the figure: T—tumor cell aggregate, EC—endothelial cell aggregate; AP—tumor cells expressing alkaline phosphotase; AP-p61-Sema3E—tumor cells expressing p61-Sema3E fused with alkaline phosphotase; p-61 Sema3E—tumor cells expressing p61-Sema3E).

### DETAILED DESCRIPTION OF THE INVENTION

The present invention provides a method for prevention of progression of an invasive disease in an individual, wherein invasion of cells, other organisms or invasion of itself plays a role in disease pathogenesis.

1. Invasive Disease

In the present content by “invasion” is meant the ability to invade tissues. This encompasses mechanisms for colonisation (adherence and initial multiplication), ability to bypass or overcame host mechanisms, and the production of extracellular substances which facilitate the actual invasive process.

The “invasive disease” of the invention is a disease, which is selected from the group comprising infectious, autoimmune, growth disturbance, neurological, cardiovascular, respiratory, kidney or neoplastic diseases.

The infectious disease of the present invention may be a disease selected from the group comprising tuberculosis, sepsis, HIV/AIDS, intestinal infectious diseases, meningitis, encephalitis, mycoses, or parasitic diseases.

The autoimmune disease of the present invention may be a disease selected from the group comprising rheumatism, lupus erythematosus, systemic sclerosis, acrosclerosis, CRST syndrome, scleroderma, or rheumatic arthritis.

By the term “autoimmune diseases” or “infectious disease” is meant a disease that has not characteristics of cancer as said characteristics are defined below.

By the terms “growth disturbance, neurological, cardiovascular, respiratory or kidney disease” the invention means any disease that is described in regular medical literature as a growth disturbance, neurological, cardiovascular, respiratory or kidney disease.

By the term “neoplastic disease” is meant a disease associated with new growth of any body tissue. A neoplastic tissue according to the invention may remain some characteristics of the tissue from which it arises but having distinct biochemical differences from the parent cell. The tissue formed due to neoplastic growth is a mutant version of the original tissue and appears to serve no physiologic function in the same sense as did the original tissue. It may be benign or malignant.

In one embodiment the invention relates to benign neoplastic disease. In another embodiment the neoplastic disease of the present invention is malignant. The malignant neoplastic disease in the present content is cancer.

The cancer may be cancer of lung, blood, breast, prostate, ovary, brain, kidney, liver, bladder, uterus, haemopoietic tissue, metabolic or endocrine system, epithelia, muscle, bone, or cancer of unknown origin. In a preferred embodiment the cancer of the invention is lung cancer.

Cancer is defined herein as a disease characterised by the presence of cancer cells which are defined in the present content by two heritable properties: they and their progeny are able (1) to reproduce unrestrained in defiance of the normal restrains (that is, they are neoplastic) and (2) invade and colonise territories normally reserved for other cells (that is, they are malignant). Invasiveness of cancer cells usually implies an ability to break loose, enter the bloodstream or lymphatic vessels, and form secondary tumors, or metastases at the other distant sites in the body.

2. The Semaphorin Family of Proteins

The present invention in one embodiment relates to a method for prevention of progression of an invasive disease, wherein invasion of cells, other organisms or invasion of itself plays a role in disease pathogenesis comprising inhibiting expression of a polypeptide belonging to the semaphorin family of proteins.

The semaphorin family of proteins (further termed “semaphorins”) is a family of bidirectional guidance factors steering axonal growth during embryogenesis, and inhibiting axonal regeneration after CNS injury. At present all semaphorins divided into 8 subclasses depending on overall structural characteristics and according to phylogenic characteristics. All semaphorins contain an ˜500-amino acid extracellular domain termed a semaphorin (sema) domain and a subclass specific C-terminus that may contain additional sequence motifs. Semaphorins also differ with respect to membrane anchorage (secreted, transmembrane, and glycosylphosphatidylinositol [GPI]-linked). The Semaphorin Nomenclature is defined in Cell 97:551-552, 1999.

In the present content by “polypeptide belonging to the semaphorin family of proteins” is meant a polypeptide, the amino acid sequence of which has at least about an 50%, at least about an 60%, at least about an 70%, at least about an 80%, more preferably at least about an 90%, even more preferably at least about an 95%, and the most preferably at about an 97% identity with the amino acid sequence of a polypeptide which is encoded by any of the DNA sequences depicted in Genbank SEQ ID NOS: X85993, L26081, U28369, X85990, X85994, AB000220, U28240, Z80941, Z91947, Z93948 AB002329, U33920, U38276, X85991, X85992, S79463, U69535, U60800, AF073289, NM_{—}004263, AF134918, U52840, X97817, X97818, AF030430, AF036585, AF069493 or AF030698, in a preferred embodiment a polypeptide, the amino acid sequence of which has at least about an 50%, at least about an 60%, at least about an 70%, at least about an 80%, more preferably at least about an 90%, even more preferably at least about an 95%, and the most preferably at about an 97% identity with the amino acid sequence of a polypeptide is encoded by any of the DNA sequences depicted in Genbank SEQ ID NOS: X85993, L26081, U28369, X85990, X85994, AB000220, U28240, Z80941, Z91947, Z93948, AB002329, or U33920, in a more preferred embodiment a polypeptide, the amino acid sequence of which has at least about an 50%, at least about an 60%, at least about an 70%, at least about an 80%, more preferably at least about an 90%, even more preferably at least about an 95%, and the most preferably at about an 97% identity with the amino acid sequence of a polypeptide is encoded by any of the DNA sequences depicted in Genbank SEQ ID NOS: Z80941, Z91947, Z93948 or NM012431, and in the most preferred embodiment a polypeptide, the amino acid sequence of which has at least about an 50%, at least about an 60%, at least about an 70%, at least about an 80%, more preferably at least about an 90%, even more preferably at least about an 95%, and the most preferably at about an 97% identity with the amino acid sequence set forth in SEQ ID NO: 1 or 2, or fagments, or natural or syntetic variants of thereof.

3. Antisense Compound

According to the present invention a method for prevention of progression of an invasive disease in an individual, wherein invasion of cells, other organisms or invasion of itself plays a role in disease pathogenesis comprises, in one aspect, inhibiting expression of a polypeptide belonging to the semaphorin family of proteins by administering to said individual an agent capable of said inhibition.

According to the present invention the agent capable of inhibiting expression of a polypeptide belonging to the semaphorin family of proteins is in one embodiment an antisense compound.

In the present content by “antisense compound” is meant an oligomeric compound, particularly an antisense oligonucleotide for use in modulating the function of nucleic acid molecules encoding the semaphorin of the invention, ultimately modulating the amount of said semaphorin produced in tumor cells. This is accomplished by providing antisense or other oligonucleotide compounds which specifically hybridize with one or more nucleic acids encoding the semaphorins as said nucleic acids defined above. As used herein, the terms “target nucleic acid” and “nucleic acid encoding a polypeptide of the semaphorin family of proteins” encompass DNA encoding said polypeptide, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of semaphorin of the invention. In the context of the present invention, “modulation” means either an increase (stimulation) or a decrease (inhibition) in the expression of a gene. In the context of the present invention, inhibition is the preferred form of modulation of gene expression, and mRNA is a preferred target It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding semaphorin of the invention. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding semaphorin of the invention, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such a mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′ most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many may contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen or reversed Hoogsteen hydrogen bonding, between complementary nucleoside or nucleotide bases. Terms “specifically hybridizable” and “complementary” are the terms, which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

The antisense compound in accordance with this invention preferably comprise from about 8 to about 50 nucleobases (i.e. from about 8 to about 50 linked nucleosides), and even more preferably from about 12 to about 30 nucleobases. In the most preferred embodiment the antisense compound is any 21 nucleotide sequence derived from a nucleic acid sequence encoding a semaphorin polypeptide (siRNA), starting with two adenines (AA) in the 5′, even more preferably those 21 nucleotide sequences starting with AA in the 5′, extending to 19 nucleobases and stopping with two thymines (TT) in the 3′ on the sense strand. Selection of the sequence of siRNA is preferably done as the following: (1) identification the starting point 75 nucleobases downstream from the start codon; (2) finding the first AA dimer; (3) recording the next 19 nucleotides following the AA dimer, (4) calculating the percentage of guanosines and cytidines (GIC content) of the AA-N₁₉ 21-base sequence, wherein a preferred G/C ratio being between about 70% and about 30%, the most preferably is about 50%; (5) the 21-nucleobase sequence is subjected to a BLAST-search (NCBI database) against EST libraries to ensure that only one gene is targeted; (6) if the conditions in either step 4 or 5 are not met, steps 2 to 5 are to be repeated. The siRNA is introduced into cells as synthetic duplex RNA consisting of the chosen 19 nucleotide sequence in sense and antisense direction, each strand contains an 3′ overhang of either UU or dTdT. The described method is disclosed in Hammondet al., 2001, Nature: 110-119; Sharp, 2001,Genes Dev 15:485-490; Elbashir et al., 2001, Genes Dev 15:188-200; Elbashiret al., 2001 Nature 411:494-498; Tuschl et al., 1999, Genes Dev 13:3191-3197; Zamore et al., 2000, Cell 101:25-33.

While antisense oligonucleotides are a preferred form of antisense compound, the present invention comprehends other oligomeric compounds, including but not limited to oligonucleotide mimetics such as are described below.

The present invention is also intended to comprehend other oligomeric compounds from about 8 to about 50 nucleobases in length which hybridize to the nucleic acid target and which inhibit expression of the target. Such compounds include ribozymes, external guide sequence (EGS) oligonucleotides (oligozymes), and other short catalytic RNAs or catalytic oligonucleotides.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred compounds useful in this invention include antisense oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA) (Nielsen et al. 1991, Science 254:1497-1500). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases are well known in art. Detailed descriptions as well as practical advice for application of modified nucleobases can be found, for example, in The Concise Encyclopedia Of Polymer Science And Engineering, p. 858-859, Kroschwitz ed. John Wiley & Sons, 1990, or in Antisense Research and Applications, Chapter 15:289-302, Crooke and Lebleu eds. CRC Press, 1993.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., 1989, Proc Natl Acad Sci USA 86:6553-56), cholic acid (Manoharan et al., 1994, Bioorg Med Chem Let 4:1053-60), a thioether, e.g., hexyl-S-tritylthiol (Manoharan et al., 1992, Ann NY Acad Sci 660:306-9; Manoharan et al., 1993, Bioorg Med Chem Le 3:2765-70), a thiocholesterol (Oberhauser et al., 1992, Nucl Acids Res 20:533-8), an aliphatic chain, e.g., dodecandiol or undecyl residues (Saison-Behmoaras et al., 1991, EMBO J 10:1111-8; Kabanov et al., 1990, FEBS Lett 259:327-330; Svinarchuk et al., 1993, Biochimie 75:49-54), a phospholipid, e.g., di-hexadecylrac-glycerol or triethyl-ammonium 1,2-di-O-hexadecyl-rac-glycero-3-H-phosphonate (Manoharan et al., 1995, Tetrahedron Left 36:3651-54; Shea et al., 1990, Nucl Acids Res 18:3777-83), a polyamine or a polyethylene glycol chain (Manoharan et al., 1995, Nucleosides & Nucleotides 14:969-973), or adamantane acetic acid (Manoharan et al., 1995,Tetrahedron Lett 36:3651-54), a palmityl moiety (Mishra et al., 1995, Biochim Biophys Acta 1264:229-237), or an octadecylamine or hexylamino-carbonyl-oxycholesterol moiety (Crooke et al., 1996, J Pharmacol Exp Ther 277:923-937).

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers.

The compounds in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules.

4. Proteolytic Processing

In another embodiment the present invention provides a method for prevention of progression of an invasive disease in an individual, wherein invasion of cells, other organisms or invasion of itself plays a role in disease pathogenesis, comprising administering to said individual an agent capable of inhibiting intracellular or extracellular proteolytic processing of a polypeptide belonging to the semaphorin family of proteins.

In vivo polypeptides are often synthesised as inactive protein precursors from which the active molecules have to be liberated by proteolytic cleavage, in particular this concerns proteins which execute their functions extracellularly. Functional proteolytic cleavage of inactive precursors may take place intracellularly by enzymes of the trans Golgi network or secretory vesicles (that is, proteolytic processing is “intracellular”), or occurs in the extracellular fluid after the protein has been secreted (that is, proteolytic processing is “extracellular”). Many enzymes that cleave the protein precursors belong to the family pro-protein convertases, which is a subfamily of serine proteases.

The polypeptide of the invention belongs to the subclass 3 secreted semaphorins defined according to the Semaphorin Nomenclature, in a more preferred embodiment to the subclass 3E, and in the most preferred embodiment is mouse Sema3E having the sequence set forth in the SEQ ID NO:1, or human SEMA3E having the sequence set forth in SEQ ID NO:2, or variants or fragments thereof.

In the present context to acquire an “active state” semaphorin of the invention requires proteolytic processing of a polypeptide belonging to the subclass 3E semaphorins by any endopeptidase, wherein said processing is occured intracellularly or extracellularly and lead to the production of a biologically active peptide fragment of said semaphorin.

By the term “biologically active peptide fragment” in the present context is meant a naturally occurring variant, proteolytic fragment or synthetic peptide comprising at least one of the following features:
(i) having at least about an 50%, more preferably at least about an 60%, more preferably at least about an 70%, more preferably at least about an 80%, more preferably at least about an 90%, even more preferably at least about an 95%, and the most preferably at about an 97% identity with the amino acid sequence set forth in SEQ ID NO: 1 or SEQ ID NO: 2, or natural or synthetic variants of thereof,
(ii) being an attractant for the lymph node and/or lung microcapillary endothelial cells,
(iii) being capable of binding and thereby activating the receptor(s) for secreted class 3 semaphorins,
(iv) promotes experimental metastases by at least 10% more as compared to a control, for example, as measured as overall weight of the tissue comprising metastasis and/or the number of tumor cells surviving after the predetermined time period,
   preferably at least two of the above features, more preferably at least three of the above features, the most preferably at least four of the above features.

The biologically active fragment of the invention is, for example, a proteolytic fragment of Sema3E and/or SEMA3E, such as an N-terminal fragment of about 61 kDa as defined by SDS-PADE under reduced conditions having the sequence

| | |
|---|---|
| MAPAGHILTLLLWGHLLELWTPGHSANPSYPRLRLSHKELLELNRTSIFQSPLG | (SEQ ID NO: 3) |
| FLDLHTMLLDEYQERLFVGGRDLVYSLNLERVSDGYREIYWPSTAVKVEECIMKG | |
| KDANECANYIRVLHHYNRTHLLTCATGAFDPHCAFIRVGHHSEEPLFHLESHR | |
| SERGRGRCPFDPNSSFVSTLVGNELFAGLYSDYWGRDSAIFRSMGKLGHIRTEH | |
| DDERLLKEPKFVGSYMIPDNEDRDDNKMYFFFTEKALEAENNAHTIYTRVGRLCV | |
| NDMGGQRILVNKWSTFLKARLVCSVPGMNGIDTYFDELEDVFLLPTRDPKNPVIF | |
| GLFNTTSNIFRGHAVCVYHMSSIREAFNGPYAHKEGPEYHWSLYEGKVPYPRPG | |
| SCASKVNGGKYGTTKDYPDDAIRFARIDPLMYQPIKPVHKKPILVKTDGKYNLR | |
| QLAVDRVEAEDGQYDVLFIGTDTGIVLKVITIYNQETEWMEEVILEELQIFKDPAPI | |
| ISMEISSKRQQLYIGSASAVAQVRFHHCDMYGSACADCCLARDPYCAWDGISCS | |
| RYYPTGAHAKRRFRR | |
| or | |
| MASAGHIITL LLWGYLLELW TGGHTADTTH PRLRLSHKEL LNLNRTSIFH | (SEQ ID NO: 4) |
| SPFGFLDLHT MLLDEYQERL FVGGRDLVYS LSLERISDGY KEIHWPSTAL | |
| KMEECIMKGK DAGECANYVR VLHHYNRTHL LTCGTGAFDP VCAFIRVGYH | |
| LEDPLFHLES PRSERGRGRC PFDPSSSFIS TLIGSELFAG LYSDYWSRDA | |
| AIFRSMGRLA HIRTEHDDER LLKEPKFVGS YMIPDNEDRD DNKVYFFFTE | |
| KALEAENNAH AIYTRVGRLC VNDVGGQRIL VNKWSTFLKA RLVCSVPGMN | |
| GIDTYFDELE DVFLLPTRDH KNPVIFGLFN TTSNIFRGHA ICVYHMSSIR | |
| AAFNGPYAHK EGPEYHWSVY EGKVPYPRPG SCASKVNGGR YGTTKDYPDD | |
| AIRFARSHPL MYQAIKPAHK KPILVKTDGK YNLKQIAVDR VEAEDGQYDV | |
| LFIGTDNGIV LKVITIYNQE MESMEEVILE ELQIFKDPVP IISMEISSKR | |
| QQLYIGSASA VAQVRFHHCD MYGSACADCC LARDPYCAWD GISCSRYYPT | |
| GTHAKRRFRR | |

or variants or homologues thereof.

A “homologous” polypeptide is defined in the present context as a polypeptide comprising at least one of the following features: (i) having an amino acid sequence which differs by at most 50 amino acids, preferably by at most 40 amino acids, preferably by at most 30 amino acids, preferably by at most 20 amino acids, preferably by at most 10 amino acids, preferably by at most 5 amino acids, more preferably by at most 3 amino acids, even more preferably by at most 2 amino acids, and most preferably by at most 1 amino acid from the amino acid sequence set forth in SEQ ID NO:3 or SEQ ID NO:4, (ii) being an attractant for the lymph node and/or lung microcapillary endothelial cells, (iii) being capable of binding and thereby activating the receptor(s) for secreted class 3 semaphorins, and (iiii) being capable of promoting experimental metastases by at least 10% more as compared to control, for example, as measured as overall weight of the tissue comprising metastasis and/or the number of tumor cells surviving after the predetermined time period, more preferably at least two of the above features, even more preferably at least tree of the above features, and the most preferably at least four of the above features.

The degree of identity between two or more amino acid sequences may be determined by means of computer programs known in the art such as GAP provided in the GCG program package (Needleman and Wunsch, 1970, J Mol Biol 48:443-453). For purposes of determining the degree of identity between two amino acid sequences for the present invention, GAP is used with the following settings: GAP creation penalty of 3.0 and GAP extension penalty of 0.1.

The amino acid sequences of the homologous polypeptides differ from the amino acid sequence set forth in SEQ ID NO:3 or SEQ ID NO:4 by an insertion or deletion of one or more amino acid residues and/or the substitution of one or more amino acid residues by different amino acid residues. Preferably, amino acid changes are of a minor nature, that is, conservative amino acid substitutions that do not significantly affect the folding and/or activity of the polypeptide; small deletions, typically of one to about 20 amino acids; small amino- or carboxyl-terminal extensions, such as an amino-terminal methionine residue; a small linker peptide of up to about 2-20 residues; or a small extension that facilitates purification by changing net charge or another function, such as a polyhistidine tract, an antigenic epitope or a binding domain.

Examples of conservative substitutions are within the group of basic amino acids (such as arginine, lysine and histidine), acidic amino acids (such as glutamic acid and aspartic acid), polar amino acids (such as glutamine and asparagine), hydrophobic amino acids (such as leucine, isoleucine and valine), aromatic amino acids (such as phenylalanine, tryptophan and tyrosine) and small amino acids (such as glycine, alanine, serine, threonine and methionine). Amino acid substitutions which do not generally alter the specific activity are known in the art and are described, e.g., by H. Neurath and R. L. Hill, 1979, in, The Proteins, Academic Press, New York. The most commonly occurring exchanges are: Ala/Ser, Val/IIe, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/IIe, Leu/Val, Ala/Glu, Asp/Gly as well as these in reverse.

The biologically active fragment of the invention is a product of the proteolytic processing of a secreted subclass 3 semaphorin on a cleavage site having the motif RXRR, wherein X may be any amino acid residue, in a more preferred embodiment S or T, in the most preferred embodiment F.

The semaphorin of the invention is cleaved by a serine protease, in a more preferred embodiment by a pro-protein convertase selected from the group comprising PC1/PC3, PC2, PC4, PC5/PC6, PC7/PC8, PACE4, or furin, and in the most preferred embodiment by furin.

Pro-protein convertases (PCs) listed above are well known in art in association with invasive diseases, in particular, see, for example, Bassi et al., 2000, Mol Carcinogen 28:63-69 for the role of PCs in cancer, or Jean et al., 2000, Proc Natl Acad Sci USA, 97:2864-9 for viral infection. Inhibition of PCs, for example, furin has been shown results in the absent or decreased invasiveness and tumorigenicity of human cancer cells in culture (Bassi et al., 2001, Proc. Natl. Acad. Sci. USA 98:10326-10331; Mercapide et al., 2002, Clin. Cancer Res. 8:1740-6) or in decrease of the production of infectious human cytomegalovirus in cell culture (Jean et al, 2000, Proc Natl Acad Sci USA, 97:2864-9). Inhibitors of a furin-like proteolytic activity are dicslosed in the above citations or in U.S. Pat. Nos. 6,022,855, 5,604,201, WO9951624 or WO9416073. However, the application of these compounds for in vivo use, as, for example, in the course of treatment of a human patient with cancer or infectious disease may be limited, as they will inhibit not only the production of pathogenic molecules, but also maturation of those factors, activity of which is necessary for the defence mechanisms of said patient and are dependent on the activity of PCs.

It is another aspect of the invention to provide new compounds directed to inhibition of PC-related proteolytic activity, of which
(i) a compound inhibiting a PC-like proteolytic activity;
(ii) a compound specifically inhibiting proteolytic processing a polypeptide belonging to the semaphorin family of proteins by a PC-like protease.

In yet another aspect the invention provides a method for producing an attractant polypeptide by establishing a cleavage product from a repulsive polypeptide, said repulsive polypeptide being a semaphorin of the invention. In a preferred embodiment said attractant is a polypeptide which can be characterised by at least one of the following features
(i) having at least about an 50%, more preferably at least about an 60%, more preferably at least about an 70%, more preferably at least about an 80%, more preferably at least about an 90%, even more preferably at least about an 95%, and the most preferably at about an 97% identity with the amino acid sequence set forth in SEQ ID NO: 3 or SEQ ID NO: 4, or natural or synthetic variants of thereof,
(ii) being an attractant for the lymph node and/or lung microcapillary endothelial cells,
(iii) being capable of binding and thereby activating the receptor(s) for secreted class 3 semaphorins.

The attractant polypeptide of the invention may be prepared by any conventional method described below for production of other polypeptides of the invention.

4.1. Inhibition of a Pro-Protein Convertase-Like Enzymatic Activity by a Peptide Fragment of a Semaphorin Polypeptide

According to the invention a method for prevention of progression of an invasive disease in an individual, wherein invasion of cells, other organisms or invasion of itself plays a role in disease pathogenesis comprises administering to said individual an agent capable of inhibiting intracellular or extracellular proteolytic processing of a polypeptide belonging to the semaphorin family of proteins.

In one embodiment the invention to provide a compound directed to inhibition of a PC-like proteolitic activity. According to the invention the compound directed to inhibition of a PC-like proteolitic activity is a peptide fragment of a semaphorin.

By the “peptide fragment of a semaphorin” in the present context is meant any peptide fragment derived from the amino acid sequence set forth in SEQ ID NO: 1 or 2, fragments, or variants of thereof, capable of inhibiting the activity an proteolityc enzyme assisting the production of a polypeptide of about 61 kDa as defined by SDS-PAGE under reduced conditions, wherein said polypeptide is characterised by at least one of the following features:
(i) having at least about an 50%, more preferably at least about an 60%, more preferably at least about an 70%, more preferably at least about an 80%, more preferably at least about an 90%, even more preferably at least about an 95%, and the most preferably at about an 97% identity with the amino acid sequence set forth in SEQ ID NO: 3 or SEQ ID NO: 4, or natural or synthetic variants of thereof,
(ii) being an attractant for the lymph node and/or lung microcapillary endothelial cells,
(iii) being capable of binding and thereby activating the receptor(s) for secreted class 3 semaphorins,
(iv) being capable of promoting the experimental metastases by at least 10% more as compared to a control, for example, as measured as overall weight of the tissue comprising metastasis and/or the number of tumor cells surviving after the predetermined time period,
   preferably at least two of the above features, more preferably at least three of the above features, the most preferably at least four of the above features.

According to the present invention the peptide fragment of semaphorin inhibiting a PC-like enzymatic activity in a preferred embodiment is a natural or synthetic contiguous amino acid sequence of at least 8 amino acids, such as for example a sequence of at least 12 amino acids, such as for example a sequence of at least 16 amino acids, such as for example a sequence of at least 20 amino acids, such as for example a sequence of at least 24 amino acids, such as for example a sequence of at least 28 amino acids, such as for example a sequence of at least 32 amino acids, such as for example a sequence of at least 36 amino acids, such as for example a sequence of at least 40 amino acids, such as for example a sequence of at least 44 amino acids, such as for example a sequence of at least 48 amino acids, such as for example a sequence of at least 52 amino acids, such as for example a sequence of at least 68 amino acids, such as for example a sequence of at least 84, such as for example a sequence of at least 100 amino acids, such as for example a sequence of at least 150 amino acids, such as for example a sequence of at least 200 amino acids, such as for example a sequence of at least 250 amino acids, such as for example a sequence of at least 300 amino acids, such as for example a sequence of at least 400 amino acids, such as for example a sequence of at least 500 amino acids derived from the sequence set forth in SEQ ID NO: 1 or SEQ ID NO : 2, or variants of thereof. In another preferred embodiment said peptide fragment of semaphorin comprises an amino acid sequence RXK/RR. In still another preferred embodiment said peptide fragment of semaphorin is comprising amino acid residues in a range of 30 to 50 amino acid residues of the sequence LARDPYCAWD GISCSRYYPT GTHAKRRFRR QDVRHGNAAQ QCFGQQFVGD (SEQ ID NO: 5), amino acid residues being numbered from the N-terminus of said sequence, such as 1-50 amino acid residues, for example 1-49 amino acid residues, such as 1-48 amino acid residues, for example 1-47 amino acid residues, such as 1-46 amino acid residues, for example 1-45 amino acid residues, such as 1-44 amino acid residues, for example 1-43 amino acid residues, such as 1-42 amino acid residues, for example 1-41 amino acid residues, such as 1-40 amino acid residues, for example 1-39 amino acid residues, such as 1-38 amino acid residues, for example 1-37 amino acid residues, such as 1-36 amino acid residues, for example 1-35 amino acid residues, such as 1-34 amino acid residues, for example 1-33 amino acid residues, such as 1-32 amino acid residues, for example 1-31 amino acid residues, such as 1-30 amino acid residues. In still yet another embodiment said peptide fragment of semaphorin is comprising amino acid residues in a range of 24 to 50 amino acid residues of the sequence LARDPYCAWD GISCSRYYPT GTHAKRRFRR QDVRHGNAAQ QCFGQQFVGD (SEQ ID NO:6), amino acid residues being numbered from the N-terminus of said sequence, such as 2-50 amino acid residues, for example 3-50 amino acid residues, such as 4-50 amino acid residues, for example from 5-50 amino acid residues, such as 6-50 amino acid residues, for example 7-50 amino acid residues, such as 8-50 amino acid residues, for example 9-50 amino acid residues, such as 10-50 amino acid residues, for example 11-50 amino acid residues, such as 12-50 amino acid residues, for example 13-50 amino acid residues, such as 14-50 amino acid residues, for example 15-50 amino acid residues, such as 16-50 amino acid residues, for example 17-50 amino acid residues, such as 18-50 amino acid residues, for example 19-50 amino acid residues, such as 20-50 amino acid residues, for example 21-50 amino acid residues, such as 22-50 amino acid residues, for example 23-50 amino acid residues, such as 24-50 amino acid residues.

A peptide fragment of semaphorin inhibiting a PC-like enzymatic activity of the present invention may be prepared by conventional synthetic methods, recombinant DNA technologies, or enzymatic cleavage of a semaphorin polypeptide.

The methods for synthetic production of peptides are well known in art. Detailed descriptions as well as practical advice for producing synthetic peptides may be found in Synthetic Peptides: A User's Guide (Advances in Molecular Biology), Grant G. A. ed., Oxford University Press, 2002, or in Pharmaceutical Formulation: Development of Peptides and Proteins, Frokjaer and Hovgaard eds., Taylor and Francis, 1999.

Alternatively the peptide fragments of semaphorin inhibiting a PC-like enzymatic activity may be produced by use of recombinant DNA technologies. A DNA sequence encoding semaphorin may be prepared as described below and fragmented by digestion with DNAase I according to a standard protocol (Sambrook et al., Molecular cloning: A Laboratory manual. 2 rd ed., CSHL Press, Cold Spring Harbor, N.Y., 1989).

The semaphorin of the invention may be encoded by a nucleic acid sequence having at least about an 50% identity with the nucleic acid sequence set forth in SEQ ID NOS: 7, 8, 9 or 10 more preferably at least about 60%, more preferably at least about 70%, more preferably at least about 80%, more preferably at least about 90%, even more preferably at least about 95%, and most preferably at least about 97%, as determined by agarose gel electrophoresis. The nucleic acid sequence may be of genomic, cDNA, RNA, semisynthetic, synthetic origin, or any combinations thereof.

The degree of identity between two nucleic acid sequences may be determined by means of computer programs known in the art such as GAP provided in the GCG program package (Needleman and Wunsch, 1970, J. Mol. Biol. 48:443-453). For purposes of determining the degree of identity between two nucleic acid sequences for the present invention, GAP is used with the following settings: GAP creation penalty of 5.0 and GAP extension penalty of 0.3.

Modification of the nucleic acid sequences encoding semaphorin polypeptides may be necessary for the synthesis of polypeptide sequences substantially similar to said polypeptides. The term “substantially similar” to semaphorin polypeptides refers to non-naturally occurring forms of said polypeptides. These polypeptide sequences may differ in some engineered way from the semaphorin polypeptides isolated from its native source. For example, it may be of interest to synthesise variants of the semaphorin polypeptides where the variants differ in specific activity, thermostability, pH optimum, or the like using, e.g., site-directed mutagenesis. The analogous sequence may be constructed on the basis of the nucleic acid sequences presented as SEQ ID NOS:7, 8, 9 or 10, e.g., a sub-sequence thereof, and/or by introduction of nucleotide substitutions which do not give rise to another amino acid sequence of semaphorins encoded by the nucleic acid sequences, but which corresponds to the codon usage of the host organism intended for production of the enzyme, or by introduction of nucleotide substitutions which may give rise to a different amino acid sequence. For a general description of nucleotide substitution, see, e.g., Ford et al., 1991, in Protein Expression and Purification 2:95-107.

It will be apparent to those skilled in the art that such substitutions can be made outside the regions critical to the function of the molecule and still result in an active polypeptide sequence. Amino acid residues essential to the activity of the polypeptide encoded by the isolated nucleic acid sequence of the invention, and therefore preferably not subject to substitution, may be identified according to procedures known in the art, such as site-directed mutagenesis or alanine-scanning mutagenesis (see, e.g., Cunningham and Wells, 1989, Science 244:1081-1085).

The recombinant semaphorin polypeptides may also be encoded by a nucleic acid sequence that hybridizes to a nucleic acid sequence set forth in SEQ ID NOS: 7, 8, 9 or 10 at low to high stringency conditions. Low to high stringency conditions are defined as pre-hybridization and hybridization at 42° C. in 5×SSPE, 0.3% SDS, 200 ug/ml sheared and denatured salmon sperm DNA and either 25, 35 or 50% formamide for low, medium and high stringencies, respectively. The carrier material is washed three times each for 30 minutes using 2×SSC, 0.2% SDS preferably at least at 50° C. (very low stringency), more preferably at least at 55° C. (low stringency), more preferably at least at 60° C. (medium stringency), more preferably at least at 65° C. (medium-high stringency), even more preferably at least at 70° C. (high stringency) and most preferably at least at 75° C. (very high stringency).

A DNA sequence encoding the semaphorin polypeptide may be prepared synthetically by established standard methods, e.g. the phosphoamidite method described by Beaucage and Caruthers, 1981, Tetrahedron Lett 22:1859-1869, or the method described by Matthes et al., 1984, EMBO J 3:801-805. According to the phosphoamidite method, oligonucleotides are synthesized, e.g. in an automatic DNA synthesizer, purified, annealed, ligated and cloned in suitable vectors.

The DNA sequence may also be of genomic or cDNA origin, for instance obtained by preparing a genomic or cDNA library and screening for DNA sequences coding for all or part of a semaphorin polypeptide by hybridization using synthetic oligonucleotide probes in accordance with standard techniques (cf. Sambrook et al., Molecular Cloning: A Laboratory Manual, 2nd Ed., Cold Spring Harbor, 1989). The DNA sequence may also be prepared by polymerase chain reaction using specific primers, for instance as described in U.S. Pat. No. 4,683,202 or Saiki et al., 1988, Science 239:487-491.

The DNA sequence is then inserted into a recombinant expression vector, which may be any vector, which may conveniently be subjected to recombinant DNA procedures. The choice of vector will often depend on the host cell into which it is to be introduced. Thus, the vector may be an autonomously replicating vector, i.e. a vector that exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

In the vector, a DNA sequence encoding semaphrin polypeptides should be operably connected to a suitable promoter sequence. The term “operatively linked” refers to the positioning of an expression control sequence with respect to a coding DNA sequence of interest such that the expression control sequence controls and regulates the transcription and translation of that DNA sequence.

The term “operatively linked” includes having an appropriate start signal (e. g., ATG) in front of the DNA sequence to be expressed and maintaining the correct reading frame to permit expression of the DNA sequence under the control of the expression control sequence and production of the desired product encoded by the DNA sequence. If a gene or DNA sequence that one desires to insert into a recombinant DNA molecule does not contain an appropriate start signal, such a start signal can be inserted in front of the gene.

The choice of expression control sequence depends upon the nature of the recombinant DNA molecule, the host that will be transformed by that recombinant DNA molecule, and whether constitutive or inducible expression of a DNA sequence of this invention is desired. Such useful expression control sequences include, for example, the early and late promoters of SV40 or adenovirus, the lac system, the trp system, the TAC or TRC system, the major operator and promoter regions of phage A, the control regions of fd coat protein, the promoter for 3-phosphoglycerate kinase or other glycolytic enzymes, the promoters of acid phosphatase (e. g., Pho5), the promoters of the yeast a-mating factors, and other sequences known to control the expression of genes of prokaryotic or eukaryotic cells or their viruses, and various combinations thereof.

Preferably the vector also encodes a selectable marker, for example, antibiotic resistance.

Replicable expression vectors can be plasmids, bacteriophages, cosmids and viruses. Any expression vector comprising RNA is also contemplated.

Preferred vectors of the present invention are derived from eukaryotic sources. Expression vectors that function in tissue culture cells are especially useful, but yeast vectors may be also contemplated. These vectors include yeast plasmids and minichromosomes, retrovirus vectors, BPV (bovine papilloma virus) vectors, baculo-virus vectors, SV40 based vectors and other viral vectors. SV40-based vectors and retrovirus vectors (e. g., murine leukemia viral vectors) are preferred. Tissue culture cells that are used with eukaryotic replicable expression vectors include Sf21 cells, CV-1 cells, COS-1 cells, NIH3T3 cells, mouse L cells, HeLa cells and such other cultured cell lines known to one skilled in the art.

In one embodiment of the invention baculovirus expression system may also be used to produce large amounts of semaphorin polypeptides in cultured insect cells. The post-translational processing-of polypeptides produced in such insect cells is similar to that of mammalian cells. Production of polypeptides in insects may therefore be advantageous.

Methods for producing polypeptides in the baculovirus expression system are known to the skilled artisan. See for example Miller, 1988, Ann Rev Microbiol 42:177. In general, a modified Autographia californica nuclear polyhedrosis virus propagated in Sf21 cells is used for polypeptide expression. This modified virus is produced by cotransfection of a small transfer vector, encoding a semaphorin polypeptide, with a viral expression vector, which has been linearized within an essential gene. Once inside the cell, the linearized expression vector can undergo recombination with the transfer vector or simply recircularize. However, only recombination gives rise to viable viruses because the function of the essential gene is lost by recircularization. Recombinant expression viruses are detected by formation of plaques.

According to the invention sequence elements capable of effecting expression of a semaphorin gene include promoters, enhancer elements, transcription termination signals and polyadenylation signal. Promotors are DNA sequence elements for controlling gene expression, in particular, they specify transcription initiation sites. Prokaryotic promoters that are useful include the lac promoter, the trp promoter, and PL and Pn promoters of lambda and the T7 polymerase promoter. Eukaryotic promoters are especially useful in the invention and include promoters of viral origin, such as the SV40 late promoter and the Moloney Leukemia virus LTR, Murine Sarcoma Virus (MSV) LTR, yeast promoters and any promoters or variations of promoters designed to control gene expression, including genetically-engineered promoters. Control of gene expression includes the ability to regulate a gene both positively and negatively (i. e., turning gene expression on or off) to obtain the desired level of expression.

The replicable expression vectors of the present invention may be made by ligating part or all of a semaphorin polypeptide coding region in the sense or antisense orientation to the promoter and other sequence elements being used to control gene expression. This juxtapositioning of promoter and other sequence elements with a semaphorin polypeptide gene allows the production of large amounts of sense or antisense semaphorin mRNA.

Therefore, one skilled in the art has available many choices of replicable expression vectors, compatible hosts and well-known methods for making and using the vectors. Recombinant DNA methods are found in any of the standard laboratory manuals on genetic engineering.

The invention also provides host cells transformed by the recombinant DNA molecules of this invention. These hosts may include well known eukaryotic and prokaryotic hosts, such as strains of E. coli, Pseudomonas, Bacillus, Streptomvces, fungi such as yeast, and animal cells, such as WHO, RLL, B-W and L-M cells, African Green Monkey kidney cells (e. g., COS 1, COS 7, BSC1, BSC40, and BMT10), insect cells (e. g., Sf9), and human cells and plant cells in tissue culture. Eukaryotic cells may harbor the recombinant DNA molecules of this invention as an extra chromosomal element or incorporate all or part of it into the host chromosome.

It will be understood that not all vectors, expression control sequences and hosts will function equally well to replicate and/or express the DNA sequences of this invention. Neither will all hosts function equally well with the same expression system.

However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic and other selective markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e. g., their compatibility with the chosen vector, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will replicate and/or express the DNA sequences of this invention on fermentation or in large scale animal culture.

Methods for transforming cells with recombinant DNA molecules are well known in the art (see, for example, Sambrook, et al., Molecular Cloning, A Laboratory Manual, Cold Spring Harbor Laboratory Press, Cold Spring Harbor, N.Y., 1989).

Any of those methods may be employed to produce the transformed hosts of this invention. Identification of transformed hosts may be achieved by assaying for the presence of semaphorin DNAs, semaphorin RNAs or semaphorin polypeptides. Additionally, transformants may be dentified by growth in selective media. For this assay, the gene necessary for growth in selective media is cotransfected into the cell either on the same or a different recombinant DNA molecule as semaphorin DNAs and is expressible in that cell. It will, of course, be obvious that the gene for selective growth should not be present in the non-transformed cell.

The transformed hosts may be employed to produce either large quantities of semaphorin DNA sequences of this invention and/or semaphorin polypeptides encoded thereby. In order to produce large quantities of semaphorin DNAs, the host, preferably a prokaryotic host, is grown in a medium and under conditions that promote DNA replication and cell division. Any complete media routinely used to grow bacteria is suitable for this purpose. Following growth, the transformed cells are separated from the growth medium and plasmid DNA is then isolated from the cells by standard and well-known techniques. Semaphorin DNAs may then be excised from the plasmid through the use of restriction endonucleases.

When the transformed hosts of this embodiment are employed, the preferred host is a mammalian cell.

The transformed host should be grown in a medium that promotes expression of semaphorin polypeptide-encoding DNA sequences present in that host. If expression of that DNA sequences is under the control of a constitutive promoter, any standard growth medium is suitable. If semaphorin DNAs is under the control of an inducible promoter, the growth medium should be supplemented with a compound that induces expression or growth conditions should be altered so as to induce expression (i. e., change in growth temperature).

Following expression, the transformed cells are separated from the growth medium, lysed and semaphorin polypeptides are purified by standard methods. If the cells secrete semaphorin polypeptides, the proteins may be harvested directly from the media without cell lysis.

Recombinant expression of semaphorin peptide fragments inhibiting a PC-like enzymatic activity may be done following the criteria described above for expression of a full-length semaphorin protein.

To obtain recombinant semapforin peptide fragments the semaphorin coding DNA fragments may be usefully fused with a second polypeptide coding DNA sequence and a protease cleavage site sequence, giving a DNA construct encoding the fusion protein, wherein the protease cleavage site coding sequence positioned between the semapforin fragment and second polypeptide coding DNA, inserted into a recombinant expression vector, and expressed in recombinant host cells. In one embodiment, said second polypeptide selected from, but not limited by the group comprising glutathion-S-reductase, calf thymosin, bacterial thioredoxin or human ubiquitin natural or synthetic variants, or peptide fragments thereof. In another embodiment, a polypeptide sequence comprising a protease cleavage site may be the Factor Xa, with the amino acid sequence IEGR, enterokinase, with the amino acid sequence DDDDK, thrombin, with the amino acid sequence LVPR/GS, or Acharombacter lyticus, with the amino acid sequence XKX, cleavage site. In still another embodiment, the host cells may be selected from the group comprising bacterial, fungal, insect, invertebrate, or mammalian cells.

4.2. Antibody Inhibiting Proteolytic Processing of the Semaphorin Polypeptide

In another aspect of the present invention proteolytic processing of any polypeptide can also be limited or completely inhibited by other methods, for example, by using compounds, which are capable of binding to a polypeptide committed to proteolysis in such a way that the proteolytic cleavage site become hidden upon binding, or folding of the polypeptide in the area of cleavage site is affected by binding. It is yet another aspect of the invention to provide a compound which inhibits proteolytic processing a semaphorin polypeptide of the invention upon binding to said polypeptide. The invention provides antibodies or fragments of antibodies directed to a semaphorin polypeptide which recognise en epitope located within the structural domain of said polypeptide, which comprises a proprotein convertase cleavage site RXK/RR, bind to this epitope and thereby inhibit proteolytic processing of said semaphorin polypeptide.

Antibody molecules belong to a family of plasma proteins called immunoglobulins, whose basic building block, the immunoglobulin fold or domain, is used in various forms in many molecules of the immune system and other biological recognition systems. A typical immunoglobulin has four polypeptide chains, containing an antigen binding region known as a variable region and a non-varying region known as the constant region.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82, 4592-4596 (1985).

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely a adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigenbinding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody which includes the variable domain complementarity determining regions (CDR), and the like forms, all of which fall under the broad term “antibody”, as used herein. The present invention contemplates the use of any specificity of an antibody, polydonal or monoclonal, and is not limited to antibodies that recognize and immunoreact with a specific antigen. In preferred embodiments, in the context of both the therapeutic and screening methods described below, an antibody or fragment thereof is used that is immunospecific for an antigen or epitope of the invention.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments that are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

Antibody fragments may be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more. In general, an antibody fragment of the invention can have any upper size limit so long as it is has similar or immunological properties relative to antibody that binds with specificity to an epitope formed by any of SEQ ID NO:1 or 2.

Antibody fragments retain some ability to selectively bind with its antigen or receptor. Some types of antibody fragments are defined as follows:

(1) Fab is the fragment that contains a monovalent antigen-binding fragment of an antibody molecule. A Fab fragment can be produced by digestion of whole antibody with the enzyme papain to yield an intact light chain and a portion of one heavy chain.

(2) Fab′ is the fragment of an antibody molecule can be obtained by treating whole antibody with pepsin, followed by reduction, to yield an intact light chain and a portion of the heavy chain. Two Fab′ fragments are obtained per antibody molecule.

Fab′ fragments differ from Fab fragments by the addition of a few residues at the carboxyl terminus of the heavy chain CH1 domain including one or more cysteines from the antibody hinge region.

(3) (Fab′)₂ is the fragment of an antibody that can be obtained by treating whole antibody with the enzyme pepsin without subsequent reduction.

(4) F(ab′)₂ is a dimer of two Fab′ fragments held together by two disulfide bonds.

Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies 113: 269-315 Rosenburg and Moore eds. Springer-Verlag, N.Y., 1994.

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad Sci. USA 90: 6444-6448 (1993).

The preparation of polyclonal antibodies is well-known to those skilled in the art. See, for example, Green et al. 1992. Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1, which are hereby incorporated by reference.

The preparation of monoclonal antibodies likewise is conventional. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG). In: Methods in Molecular Biology, 1992, 10:79-104, Humana Press, NY.( ).

Methods of in vitro and in vivo manipulation of monoclonal antibodies are well known to those skilled in the art. For example, the monoclonal antibodies to be used in accordance with the present invention may be made by the hybridoma method first described by Kohler and Milstein, Nature 256, 495 (1975), or may be made by recombinant methods, e.g., as described in U.S. Pat. No. 4,816,567. The monoclonal antibodies for use with the present invention may also be isolated from phage antibody libraries using the techniques described in Clackson et al., 1991, Nature 352: 624-628, as well as in Marks et al., 1991, J Mol Biol 222: 581-597. Another method involves humanizing a monoclonal antibody by recombinant means to generate antibodies containing human specific and recognizable sequences. See, for review, Holmes, et al., 1997, J Immunol 158:2192-2201 and Vaswani, et al., 1998, Annals Allergy, Asthma & Immunol 81:105-115.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody indicates the character of the antibody as being obtained from a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al., 1984, Proc Natl Acad Sci 81, 6851-6855.

Methods of making antibody fragments are also known in the art (see for example, Harlow and Lane, Antibodies: A Laboratory Manual, Cold Spring Harbor Laboratory, NY, 1988, incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, in U.S. Pat. No. 4,036,945 and U.S. Pat. No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E coli. The recombinant host cells synthesize a single polypeptide chain with a linker peptide bridging the two V domains. Methods for producing sFvs are described, for example, by Whitlow, et al., 1991, In: Methods: A Companion to Methods in Enzymology, 2:97; Bird et al., 1988, Science 242:423-426; U.S. Pat. No. 4,946,778; and Pack, et al., 1993, BioTechnology 11:1271-77.

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., 1986, Nature 321, 522-525; Reichmann et al., 1988, Nature 332, 323-329; Presta, 1992, Curr Op Struct Biol 2:593-596; Holmes et al., 1997, J Immunol 158:2192-2201 and Vaswani, et al., 1998, Annals Allergy, Asthma & Immunol 81:105-115.

The generation of antibodies may be achieved by standard methods in the art for producing polyclonal and monoclonal antibodies using a natural or recombinant semaphorin polypeptide or fragment thereof as an antigen. Such antibodies would be in a preferred embodiment generated using a naturally occurring or recombinantly produced semaphorin polypeptides having an amino acid sequence set forth in SEQ ID NO: 1 or SEQ ID NO: 2, or variants or fragments thereof, or, in a more preferred embodiment, using fragments of said semaphorin polypeptides, wherein said fragments would meet at least one of the following criteria:
(i) being a natural or synthetic contiguous amino acid sequence of at least 8 amino acids, more preferably of at least 12 amino acids, such as for example a sequence of at least 16 amino acids such as for example a sequence of at least 20 amino acids, such as for example a sequence of least 24 amino acids, such as for example a sequence of at least 28 amino acids, such as for example a sequence of at least 32 amino acids, such as for example a sequence of at least 36 amino acids, such as for example a sequence of at least 40 amino acids, such as for example a sequence of at least 44 amino acids, such as for example a sequence of at least 48 amino acids, such as for example a sequence of at least 52 amino acids, such as for example a sequence of at least 68 amino acids, such as for example a sequence of at least 84, such as for example a sequence of at least 100 amino acids, such as for example a sequence of at least 150 amino acids, such as for example a sequence of at least 200 amino acids, such as for example a sequence of at least 250 amino acids, such as for example a sequence of at least 300 amino acids, such as for example a sequence of at least 400 amino acids, such as for example a sequence of at least 500 amino acids derived from the sequence set forth in SEQ ID NO: 1 or SEQ ID NO: 2, or variants of thereof;
(ii) comprising an amino acid sequence RXK/RR, wherein X may be any amino acid residue, in a more preferred embodiment S or T, in the most preferred embodiment F;
(iii) comprising amino acid residues in a range of 30 to 50 amino acid residues of the sequence LARDPYCAWD GISCSRYYPT GTHAKRRFRR QDVRHGNAAQ QCFGQQFVGD (SEQ ID NO:4), amino acid residues being numbered from the N-terminus of said sequence, such as 1-50 amino acid residues, for example 1-49 amino acid residues, such as 1-48, for example 1-47 amino acid residues, such as 1-46 amino acid residues, for example 1-45 amino acid residues, such as 1-44 amino acid residues, for example 1-43 amino acid residuess, such as 1-42 amino acid residuess, for example 1-41 amino acid residues, such as 1-40 amino acid residues, for example 1-39 amino acid residues, such as 1-38 amino acid residues, for example 1-37 amino acid residues, such as 1-36 amino acid residues, for example 1-35 amino acid residues, such as 1-34 amino acid residues, for example 1-33 amino acid residues, such as 1-32 amino acid residues, for example 1-31 amino acid residues, such as 1-30 amino acid residues;
(iv) comprising amino acid residues in a range of 24 to 50 amino acid residues of the sequence LARDPYCAWD GISCSRYYPT GTHAKRRFRR QDVRHGNAAQ QCFGQQFVGD (SEQ ID NO:5), amino acid residues being numbered from the N-terminus of said sequence, such as 2-50 amino acid residues, for example 3-50 amino acid residues, such as 4-50 amino acid residues, for example from 5-50 amino acid residues, such as 6-50 amino acid residues, for example 7-50 amino acid residues, such as 8-50 amino acid residues, for example 9-50 amino acid residues, such as 10-50 amino acid residues, for example 11-50 amino acid residues, such as 12-50 amino acid residues, for example 13-50 amino acid residues, such as 14-50 amino acid residues, for example 15-50 amino acid residues, such as 16-50 amino acid residues, for example 17-50 amino acid residues, such as 18-50 amino acid residues, for example 19-50 amino acid residues, such as 20-50 amino acid residues, for example 21-50 amino acid residues, such as 22-50 amino acid residues, for example 23-50 amino acid residues, such as 24-50 amino acid residues.

In another embodiment the antibodies are produced in vivo by the individual to be treated, for example, by administering an immunogenic fragment according to the invention to said individual. Accordingly, the present invention further relates to a vaccine comprising an immunogenic fragment described above.

5. Inhibition of Activation of the Receptor.

In still another embodiment the present invention provides a method for prevention of progression of a invasive disease in an individual, wherein invasion of cells, other organisms or invasion of itself plays a role in disease pathogenesis, comprising administering to said individual an agent capable of inhibiting binding a proteolytic fragment of a polypeptide belonging to the semaphorin family of proteins to a receptor and thereby inhibiting sequential activation of said receptor.

Cells express different receptors on both the internal and external membranes. According to the invention, a proteolytic fragment of a semaphorin, which is defined above, binds to and thereby activates a receptor, which is expressed on the external membrane of the cells. These cells may, for example, be neurons, glial cells, all type of muscle cells, neuroendocrine cells, gonadal cells, kidney cells, liver cells, blood cells, fibroblasts or endothelial cells. In a preferred embodiment the cells are endothelial cells. In a more preferred embodiment, the cells are lymph node, lung or bone microcapillary cells. In another preferred embodiment, cells may be tumor cells, which express the receptor to the above described proteolytic fragment of a semaphorin, which said cells produce themselves.

In the present context the term “binds” refers to the direct or indirect contact between a semaphorin proteolytic fragment of the invention and a receptor, preferably a direct interaction. The term “direct interaction” means that the compound in question binds directly to the receptor. The term “indirect” means that the compound binds to another molecule(s) that is associated with the receptor in such a way that the binding of said compound to said molecule(s) leads to sequential activation of the receptor. The invention defines said association of the receptor with another molecule(s) as a receptor complex. Examples of molecules that may be found in a receptor complex of the invention may be represented but not limited by the molecules belonging to the families of neuropilins, plexins, tyrosine kinase receptors, members of the immunoglobulin superfamily of adhesion molecules or proteoglycans. The invention also contemplates an assisted binding to the receptor. By the term “assisted binding” is meant that a compound is capable to activate the receptor by the direct binding which is a low affinity binding, however the presence of a molecule(s) which together with the receptor is capable to form a receptor complex increases the affinity of binding and thereby promote activating of the receptor. A molecule that may be involved in the assisted binding of the invention may be selected from the group of molecules described for the receptor complex above. A low affinity binding is defined herein as the binding having affinity of the interaction between a compound and the corresponding receptor within the range of Kd 10⁻³-10⁻¹¹M. The term “activate” refers to any change that can be registered in cells expressing the receptor upon interaction of said semaphorin proteolytic fragment and said receptor or the receptor associated molecule. This change may be a registered change of a metabolic status of a cell, for example, a selected activation or inhibition some intracellular biochemical pathways resulting in expression, degradation or modification of biological micromolecules such as, for example, proteins, nucleic acids, lipids or carbohydrates. In a preferred embodiment the change is a registered change of the cell physiological status or behaviour, such as for example, differentiation, proliferation, apoptosis or cell motility. In the most preferred embodiment, the registered change is activation of cell motility upon binding the proteolytic fragment of a semaphorin to an extracellular receptor.

At the present there are two families of receptors for the subclass 3 secreted semaphorins are known in art: Neuropilins and Plexins. According to the invention the proteolytic fragment of a semaphorin binds to a receptor belonging to the Plexin family receptors or to a receptor complex containing said receptor and thereby activates the receptor. Preferred embodiments for the receptor belonging to the Plexin family receptors concern a receptor of the Plexin A subfamily. More preferred embodiments concerns Plexin A1, or Plexin A2, or Plexin A3.

It is an additional aspect of the present invention to provide a compound capable of inhibiting binding a proteolytic fragment of a polypeptide belonging to the semaphorin family of proteins to a receptor and thereby inhibiting sequential activation of said receptor. In the present context the compound may be any chemical which will prevent binding of said proteolytic fragment to said receptor and/or activation of said receptor. In a preferred embodiment, the compound may be a glucosaminoglycan, for example, heparin, heparan sulphate, chondroitin sulphate, keratin sulphate, dermatan sulphate, or their deviates, or a peptide which is capable of binding to the proteolytic fragment of a semapforin and thereby limiting the binding capacity of said fragment to the Plexin A1, or Plexin A2, or Plexin A3 receptor, or a peptide, which is capable of interacting with the binding site for said fragment on the molecule of the Plexin A1, or Plexin A2, or Plexin A3 receptor without activation of said receptors and thereby inhibiting binding the proteolytic fragment of a semaphorin to said receptors and sequential activation of said receptors. In the most preferred embodiment the present invention relates to a compound, which is
(i) a peptide fragment of a semaphorin comprising a sequence set forth in SEQ ID NO: 1 or SEQ ID NO: 2, which is capable of binding the Plexin A1 receptor without consequent activation of said receptor. In a preferred embodiment, said peptide fragment is a contiguous sequence of at least about 8 amino acids derived from the amino sequence
MAPAGHILTLLLWGHLLELWTPGHSANPSYPRLRLSHKELLELNRT- SIFQSPLGFLDLHTMLLDEYQERLFVGGRDLVYSLNLERVSDGYREIYWP- STAVKVEECIMKGKDANECANYIRVLHHYNRTHLLTCATGAFDPHCAFIR- VGHHSEEPLFHLESHRSERGRGRCPFDPNSSFVSTLVGNELFAGLYSDY- WGRDSAIFRSMGKLGHIRTEHDDERLLKEPKFVGSYMIPDNEDRDDNK- MYFFFTEKALEAENNAHTIYTRVGRLCVNDMGGQRILVNKWSTFLKARL- VCSVPGMNGIDTYFDELEDVFLLPTRDPKNPVIFGLFNTTSNIFR- GHAVCVYHMSSIREAFNGPYAHKEGPEYHWSLYEGKVPYPRPG- SCASKVNGGKYGTTKDYPDDAIRFARIDPLMYQPIKPVHKKPILVKTDG- KYNLRQLAVDRVEAEDGQYDVLFIGTDTGIVLKVITIYNQETEWMEE- VILEELQIFKDPAPIISMEISSKRQQLYIGSASAVAQVRFHHCDMYGSA- CADCCLARDPYCAWDGISCSRYYPTGAHAKRRFRR (SEQ ID NO: 3), or variants thereof,
or from the amino acid sequence
MASAGHIITL LLWGYLLELW TGGHTADTTH PRLRLSHKEL LNLNRTSIFH SPFGFLDLHT MLLDEYQERL FVGGRDLVYS LSLERISDGY KEIHWPSTAL KMEECIMKGK DAGECANYVR VLHHYNRTHL LTCGTGAFDP VCAFIRVGYH LEDPLFHLES PRSERGRGRC PFDPSSSFIS TLIGSELFAG LYSDYWSRDA AIFRSMGRLA HIRTEHDDER LLKEPKFVGS YMIPDNEDRD DNKVYFFFTE KALEAENNAH AIYTRVGRLC VNDVGGQRIL VNKWSTFLKA RLVCSVPGMN GIDTYFDELE DVFLLPTRDH KNPVIFGLFN TTSNIFRGHA ICVYHMSSIR AAFNGPYAHK EGPEYHWSVY EGKVPYPRPG SCASKVNGGR YGTTKDYPDD AIRFARSHPL MYQAIKPAHK KPILVKTDGK YNLKQIAVDR VEAEDGQYDV LFIGTDNGIV LKVITIYNQE MESMEEVILE ELQIFKDPVP IISMEISSKR QQLYIGSASA VAQVRFHHCD MYGSACADCC LARDPYCAWD GISCSRYYPT GTHAKRRFRR (SEQ ID NO: 4) or variants thereof, and/or
(ii) a peptide fragment of the Plexin A1, Plexin A2 or Plexin A3 receptor capable of binding a proteolytic fragment of a semaphorin, wherein said semaphorin is having the amino acid sequence set forth in SEQ ID NO: 1 or SEQ ID NO: 2, in a more preferred embodiment, the proteolytic fragment of a semaphorin with a sequence set forth in SEQ ID NO:3 or SEQ ID NO: 4. In another preferred embodiment, the peptide fragment of Plexin A1, or Plexin A2, or Plexin A3 receptor capable of binding defined above proteolytic fragment of a semaphorin, comprising at least an 8 amino acid contiguous sequence derived from the sequence of the ectodomain of Plexin A1, comprising a sequence from 1 to 680 amino acid residue of the Plexin A1 amino acid sequence as it is defined in Genbank SEQ ID NO: X87832.2, or the ectodomain of Plexin A2 comprising a sequence from 1 to 677 amino acid residues of the Plexin A2 amino acid sequence as it is defined in Genbank SEQ ID NO: BAA32308, or the ectodomain of Plexin A3 comprising a sequence from 1 to 542 amino acid residue of the Plexin A3 amino acid sequence as it is defined in Genbank SEQ ID NO: P51805.

The peptide compounds described above may be prepared by a relevant method described for preparation of the other peptide compounds of the invention.

In a specific embodiment the invention provides a method for inhibiting activation of a Plexin receptor in vivo by a fragment of the semaphorin polypeptide of the invention derived from proteolytic processing said polypeptide by a proprotein convertase, comprising applying to cancer cells an agent capable of inhibiting proteolytic cleavage of said polypeptide by said convertase. The agent capable of inhibiting proteolytic processing of the semaphorin polypeptide may be selected from the group comprising commercially available inhibitors of serine proteases and/or proprotein convertases, such as for example decanoyl-RVKR-chloromethylketone (Bachem), glycosaminoglycans, such as for example heparin, heparan sulphate, chondroitin sulphate, keratin sulphate, dermatan sulphate, or their deviates. In a preferred embodiment, said agent is selected from the group comprising a peptide derived from a sequence of semaphoring of the invention, which is capable of interacting with the binding site for a proteolytic fragment of semaphorin on the molecule of the Plexin A1, or Plexin A2, or Plexin A3 receptor without activation of said receptor, as said peptide described above, or a peptide derived from the sequence of the Plexin A1, or Plexin A2, or Plexin A3 receptor, which is capable of binding to the proteolytic fragment of a semaphorin and thereby limiting the binding capacity of said fragment to said receptors, as said peptide described above.

6. Methods

6.1. Method for Prevention of Progression of an Invasive Disease in an Individual, Wherein Invasion of Cells, Other Organisms or Invasion of Itself Plays a Role in Disease Pathogenesis.

It is an objective of the present patent application to provide a method for prevention of progression of an invasive disease in an individual, wherein invasion of cells, other organisms, or invasion of itself plays a role in disease pathogenesis, said method being based on using of the compounds capable of inhibiting progression of an invasive disease associated with metabolism of a polypeptide belonging to the semaphorin family. To be applied in method the compounds according to the invention to be formulated as medicaments. Thus, the invention concerns the use of the compounds described above for the manufacture of a medicament for prevention of progression of an invasive disease in an individual, wherein invasion of cells, other organisms or invasion of itself plays a role in disease pathogenesis.

Thus, in first embodiment the above method may comprise (i) using of an agent capable of inhibiting expression of a polypeptide belonging to the semaphorin family of proteins for the manufacture of a medicament for prevention of progression of an invasive disease in an individual, wherein invasion of cells, other organisms or invasion of itself plays a role in disease pathogenesis.

The agent is preferably an antisense nucleotide. The most preferable antisense nucleotides of the method as described above.

In another embodiment the method may comprise (ii) using of an agent capable of inhibiting intracellular or extracellular proteolytic processing of a polypeptide belonging to the semaphorin family of proteins for the manufacture of a medicament for prevention of progression of an invasive disease in an individual, wherein invasion of cells, other organisms or invasion of itself plays a role in disease pathogenesis.

The agent is preferably selected from antibodies or fragments of antibodies directed to said polypeptide, or fragments or variants of fragments of said polypeptide. Preferable embodiments of these antibodies and variants are discussed above.

In still another embodiment the method may comprise (iii) using of an agent capable of inhibiting binding of a proteolytic fragment of a polypeptide belonging to the semaphorin family of proteins to a receptor and thereby inhibiting sequential activation of said receptor for the manufacture of a medicament for prevention of progression of an invasive disease in an individual, wherein invasion of cells, other organisms or invasion of itself plays a role in disease pathogenesis.

Preferred embodiments of the agent of step (iii) are discussed above.

The method comprising the use of any of the steps i-iii may be practised for prevention of progression of an invasive disease selected from the group comprising autoimmune, infectious, growth disturbance, neurological, cardiovascular, respiratory, kidney or neoplastic diseases, with the proviso that the disease is not cancer. Embodiments of such diseases are discussed above. The method comprising the use of any of the steps ii-iii may be practised for prevention of progression of an invasive disease selected from the group comprising neoplastic diseases. In a preferred embodiment the neoplastic disease is cancer.

In another embodiment the above method may comprise a combination of any of the steps i-iii. The combinations (i) and (ii), (i) and (iii), (i) and (ii) and (iii) (ii) and (iii) are suitable for prevention of any invasive disease selected from the group comprising autoimmune, infectious and neoplastic disease. By the term “combination” in the present context is meant that the method includes the use of at least two steps selected from steps (i), (ii) and (iii) been performed in any order, for example the step (ii) may follow the step (i), or vice versa.

6. 2. Method for Prevention of Metastases and/or Treatment of Malignant Forms of Cancer

It is another objective of the invention to provide a method for the prevention of metastases of cancer in vivo and tumor progression in vivo or in vitro. The method of invention may comprise any of the following steps
i) use of a sufficient amount of an agent capable of inhibiting intracellular or extracellular proteolytic processing of a polypeptide belonging to the semaphorin family of proteins, wherein the agent is selected from antibodies or fragments of antibodies directed to said polypeptide, or fragments or variants of fragments of said polypeptide, or
ii) use of a sufficient amount of an agent capable of inhibiting binding a proteolytic fragment of a polypeptide belonging to the semaphorin family of proteins to a receptor and thereby inhibiting sequential activation of said receptor.

To be used in the above method the compounds according to the invention to be formulated as medicaments. tumor progression in vivo or in vitro. Therefore, the invention first concerns the use of the compounds for the manufacture of a medicament for prevention of metastases of cancer in vivo.

In another embodiment the above method may comprise a combination of steps (i) and (ii) in any order, In still another embodiment the method may comprise a step of using a sufficient amount of an agent capable of inhibiting expression of a polypeptide belonging to the semaphorin family of proteins in combination with any of the steps (i) or (ii), or a combination of the all three steps performed in any order.

It is yet another objective to use the above method for the treatment of malignant forms of cancer.

The malignant forms cancer of the invention are cancers, which possess any of the malignant cancer phenotype characteristics known in medical literature. The malignant cancer of the invention is preferably selected from the group comprising carcinomas, melanomas, sarcomas, gliomas, or blastomas.

6.3. Method for Diagnosis and/or Prognosis of Malignant Cancer

Further objective of the present invention concerns providing a method for diagnosis of malignant cancer, said method comprising any of the steps
i) assessing the level of expression of semaphorin polypeptides of the invention in a tumor, and
ii) detecting fragments of the semaphorin polypeptides of the invention in a body liquid, such as blood, urea or faeces, and
iii) measuring the ratio between full length semaphorin polypeptides of the invention and peptide fragments of said semaphorins in a tumor and/or a body liquid, such as blood, urea or faeces.

The invention also concerns the use of the above method for prognosis of malignancy of cancer.

Typical embodiments of the above diagnostic and prognostic methods, which utilize semaphorin specific oligonucleotides, polypeptides, and antibodies are analogous to those methods from well-established diagnostic assays which employ, e.g., semaphorin specific oligonucleotides, polypeptides and antibodies. For example, just as semaphorin specific oligonucleotides are used as probes and primers in Northern and PCR analysis to observe the presence and/or the level of semaphorin mRNAs in methods of monitoring semaphorin overexpression or the metastatic ability of mouse tumor cell lines (Christensen et al, 1998, Cancer Res 58:1238-44), the oligonucleotide compounds of the invention can be utilized in the same way to detect the semaphorin polypeptides overexpression in a tumor or evaluate the metastatic ability of a tumor expressing this gene. Alternatively, the semaphorin polypeptides of the invention are used to generate the antibodies specific for semaphorins which can then be used to observe the presence and/or the level of semaphorin polypeptides and their peptide fragments in tissues employing methods of immunodetection well known in art.

For detecting semaphorin polypeptides of the invention and their fragments in a body liquid, such as blood, urea or faeces the methods employing the antibody compounds described above can be used. These methods are well known in art and include conventional screening methods such as, for example, immunoblotting, ELISA, or new technologies, such as, for example, ProteinChip/SELDI-TOF-MS. It is still another important embodiment of the invention to use the compounds of the invention described above for the manufacture of a kit(s) for diagnosis and/or prognosis of malignancy of a tumor.

7. Compounds

Furthermore, the invention relates in an additional preferred embodiment to providing a compound(s) for the manufacture of a medicament(s) for use in the method for

1) prevention of progression of an invasive disease, wherein invasion of cells, other organisms or invasion of itself plays a role in disease pathogenesis, and/or

2) prevention of metastases of cancer in vivo and tumor progression in vivo and in vitro, and/or

3) treatment of malignant forms of cancer and/or

4) diagnosis of malignant cancer and/or

5) prognosis of malignancy of cancer.

The compound of the invention is, in one embodiment, an antisense nucleotide at about 19 nucleobases in length comprising at least an 5-nucleobase portion of the sequence set forth in SEQ ID NOS: 7, 8, 9 or 10, which specifically binds to a nucleic acid encoding of a semaphorin polypeptide and thereby inhibits expression said polypeptide.

In another embodiment, the compound of the invention is a peptide fragment of a semaphorin having a sequence of about 8 to about 100 amino acids derived from the sequence set forth in SEQ ID NO:1 or SEQ ID NO: 2, comprising a proprotein convertase cleavage site RXK/RR, more preferably a peptide fragment of a semaphorin comprising amino acid residues in a range of 30 to 50 amino acid residues of the sequence LARDPYCAWD GISCSRYYPT GTHAKRRFRR QDVRHGNAAQ QCFGQQFVGD (SEQ ID NO:5), amino acid residues being numbered from the N-terminus of said sequence, such as 1-50 amino acid residues, for example 1-49 amino acid residues, such as 1-48, for example 1-47 amino acid residue, such as 1-46 amino acid residues, for example 1-45 amino acid residues, such as 1-44 amino acid residues, for example 1-43 amino acid residues, such as 1-42 amino acid residues, for example 1-41 amino acid residues, such as 1-40 amino acid residues, for example 1-39 amino acid residues, such as 1-38 amino acid residues, for example 1-37 amino acid residues, such as 1-36 amino acid residues, for example 1-35 amino acid residues, such as 1-34 amino acid residues, for example 1-33 amino acid residues, such as 1-32 amino acid residues, for example 1-31 amino acid residues, such as 1-30 amino acid residues, or a peptide fragment of semaphorin comprising amino acid residues in a range of 24 to 50 amino acid residues of the sequence LARDPYCAWD GISCSRYYPT GTHAKRRFRR QDVRHGNAAQ QCFGQQFVGD (SEQ ID NO:6), amino acid residues being numbered from the N-terminus of said sequence, such as 2-50 amino acid residues, for example 3-50 amino acid residues, such as 4-50, for example from 5-50 amino acid residues, such as 6-50 amino acid residues, for example 7-50 amino acid residues, such as 8-50 amino acid residues, for example 9-50 amino acid residues, such as 10-50 amino acid residues, for example 11-50 amino acid residues, such as 12-50 amino acid residues, for example 13-50 amino acid residues, such as 14-50 amino acid residues, for example 15-50 amino acid residues, such as 16-50 amino acid residues, for example 17-50 amino acid residues, such as 18-50 amino acid residues, for example 19-50 amino acid residues, such as 20-50 amino acid residues, for example 21-50, such as 22-50 amino acid residues, for example 23-50, such as 24-50 amino acid residues.

In still another embodiment, the compound of the invention is an isolated antibody or a fragment of antibody, wherein said antibody being raised against a semaphorin of the invention, or against natural or artificial variants, or peptide fragments of thereof, which specifically binds to and thereby inhibits the cleavage of said semaphorin by a serine protease in vivo, in a more preferred embodiment, said antibody recognises and binds to an epitope located within a sequence of about 5 to about 50 amino acids in length located in the structural domain of a semaphorin of the invention, which comprises a proprotein convertase cleavage site RXK/RR. The antibody of the invention may be selected from the group comprising a polyclonal antibody or monoclonal antibody.

In yet another embodiment, the compound of the invention is a peptide fragment of a semaphorin of the invention capable of inhibiting binding of a proteolytic fragment of said semaphorin to a receptor and thereby inhibiting sequential activation of said receptor. In a more preferred embodiment, said peptide fragment of semaphorin is a contiguous sequence of at least 8 amino acids derived from the amino acid sequence set forth in SEQ ID NO: 3 or SEQ ID NO:4.

In yet still another embodiment, the compound of the invention is a peptide fragment of a Plexin receptor, more preferably, a Plexin A receptor, the most preferably, the Plexin A1, or Plexin A2, or Plexin A3 receptor, having a contiguous sequence at least of about 8 amino acids derived from the sequence of ectodomain of Plexin A1 receptor comprising the sequence set forth in SEQ ID NO: 11, or natural or synthetic variants thereof, or from the sequence of ectodomain of Plexin A2 receptor comprising the sequence set forth in SEQ ID NO:12, or natural or synthetic variants thereof, or from the sequence of ectodomain of Plexin A3 receptor comprising the sequence set forth in SEQ ID NO: 13, or natural or synthetic variants thereof, capable of binding of a proteolytic fragment of a semaphorin of the invention, as said fragment defined above.

Preparation of any peptide compounds of the invention or antibody compounds may be done is as described in detail above.

In additional embodiment, the compound of the invention may be any molecule that will meet requirements described above for either the antisense, peptide or antibody compound. In a preferred embodiment, said molecule is having at least about 50% bioavailability and is selected from the group comprising acyclic, cylic or heterocyclic hydrocarbons with molcular weights (MW) from 20-1000 containing e.g. oxygen, sulphur, nitrogen, phosphor, selenium, boron, fluorine, chlorine, bromine, silicium or metal organic compounds.

8. Parmaceutical Composition

Pharmaceutical compositions containing compounds of the present invention may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications. Strategies in formulation development of medicaments and compositions based on the compounds of the present invention generally correspond to formulation strategies for any other protein or oligonucleotide-based drug product. Potential problems with therapeutic peptide formulation and delivery and the guidance required to overcome these problems are dealt with in several textbooks, e.g. “Therapeutic Peptides and Protein Formulation. Processing and Delivery Systems”, Ed. A. K. Banga, Technomic Publishing AG, Basel, 1995. The methods for pharmaceutical formulation and delivery of therapeutic oligonucleotides of the invention may be selected from those, that are, for example, described by Tavitian et al., 2002, Pharm Res 19:367-76, Agrawal et al., 2002, Int J Oncol 21:65-72, Arora et al., 2002, J Pharm Sci 91:1009-18 or Lopes and Mayer, 2002, Cancer Chemother Pharmacol 49:57-68

The main routes of drug delivery according to the present invention are pareterally, oral, or topical administation. Other drug-administration methods, which are effective to deliver the drug to a target site or to introduce the drug into the bloodstream, are also contemplated.

Compounds of the invention may be administered parenterally, that is by intravenous, intramuscular, subcutaneous, intranasal, intrarectal, intravaginal or intraperitoneal administration. The subcutaneous and intramuscular forms of parenteral administration are generally preferred. Appropriate dosage forms for such administration may be prepared by conventional techniques. The complexes may also be administered by inhalation, that is by intranasal and oral inhalation administration.

The compounds according to the invention may be administered with at least one other compound. The compounds may be administered simultaneously, either as separate formulations or combined in a unit dosage form, or administered sequentially.

Furthermore, the compounds of the invention may be administrated together with one or more other conventional cancer therapies. Conventional cancer therapy includes for example surgery, radiotherapy, chemotherapy, therapy with hormones, and immunotherapy.

Injectables are usually prepared either as liquid solutions or suspensions, solid forms suitable for solution in, or suspension in, liquid prior to injection. The preparation may also be emulsified. The active ingredient is often mixed with excipients which are pharmaceutically acceptable and compatible with the active ingredient. Suitable excipients are, for example, water, saline, dextrose, glycerol, ethanol or the like, and combinations thereof. In addition, if desired, the preparation may contain minor amounts of auxiliary substances such as wetting or emulsifying agents, pH buffering agents, or which enhance the effectiveness or transportation of the preparation.

Formulations of the compounds of the invention can be prepared by techniques known to the person skilled in the art. The formulations may contain pharmaceutically acceptable carriers and excipients including microspheres, liposomes, microcapsules, nanoparticles or the like.

The preparation may suitably be administered by injection, optionally at the site, where the active ingredient is to exert its effect. Additional formulations which are suitable for other modes of administration include suppositories, nasal, pulmonal and, in some cases, oral formulations. For suppositories, traditional binders and carriers include polyalkylene glycols or triglycerides. Such suppositories may be formed from mixtures containing the active ingredient(s) in the range of from 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and generally contain 10-95% of the active ingredient(s), preferably 25-70%.

Other formulations are such suitable for nasal and pulmonal administration, e.g. inhalators and aerosols.

The active compound may be formulated as neutral or salt forms. Pharmaceutically acceptable salts include acid addition salts (formed with the free amino groups of the peptide compound) and which are formed with inorganic acids such as, for example, hydrochloric or phosphoric acids, or such organic acids as acetic acid, oxalic acid, tartaric acid, mandelic acid, and the like. Salts formed with the free carboxyl group may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are of the order of several hundred pg active ingredient per administration with a preferred range of from about 0.1 μg to 1000 μg, such as in the range of from about 1 μg to 300 μg, and especially in the range of from about 10 μg to 50 μg. Administration may be performed once or may be followed by subsequent administrations. The dosage will also depend on the route of administration and will vary with the age and weight of the subject to be treated. A preferred dosis would be in the interval 30 mg to 70 mg per 70 kg body weight.

Some of the compounds of the present invention are sufficiently active, but for some of the others, the effect will be enhanced if the preparation further comprises pharmaceutically acceptable additives and/or carriers. Such additives and carriers will be known in the art. In some cases, it will be advantageous to include a compound, which promote delivery of the active substance to its target.

In many instances, it will be necessary to administrate the formulation multiple times. Administration may be a continuous infusion, such as intraventricular infusion or administration in more doses such as more times a day, daily, more times a week, weekly, etc. Many of the compounds exhibit a long term effect whereby administration of the compounds may be conducted with long intervals, such as 1 week or 2 weeks.

The administration may be continuous or in small portions based upon controlled release of the active compound(s). Furthermore, precursors may be used to control the rate of release and/or site of release. Other kinds of implants and well as oral administration may similarly be based upon controlled release and/or the use of precursors.

### EXAMPLES

### Example 1

### Cloning of Human and Mouse Semaphorins

A 885 bp fragment of the human SEMA3E gene was cloned by degenerate PCR using cDNA derived from human MDA-MB-468 total RNA and degenerate primers designed to bind sequences encoding semaphorin-specific amino-acid motifs: 5′CGGGATCCAT(A/C/T)TT(C/T)TT(C/T)TT(C/T)AC(A/G/C/T)GA(A/G)AA-3′ (SEQ ID NO: 14), the sense primer encoding the YFFFTEK motif, and 5′-GCGGATCCTCCCA(A/G/C/T)GC(A/G)CA(A/G)TA(A/G/C/T)GG(A/G)TC-3′ (SEQ ID NO: 15), the antisense primer complementary to the sequence encoding the DPYCAWD motif. The amplified sequence was directly cloned onto the TA cloning vector pCRII (Invitrogen).

Fragments of murine Sema3A, 3B, and 3C were isolated from day 14 embryos using RT-PCR, with the following set of primers:

| | |
|---|---|
| (sema3A antisense) | |
| 5′ACATGCACACAGCAGATCCC-3′; | (SEQ ID NO: 16) |
| (sema3A sense) | |
| 5′GGAAGAGCCCTTATGATCCC-3′; | (SEQ ID NO: 17) |
| (sema3B antisense) | |
| 5′CAACTCCAGGTACTGAGCAC-3′; | (SEQ ID NO: 18) |
| (sema3B sense) | |
| 5′AATGCAACTGGGCAGGGAAG-3′; | (SEQ ID NO: 19) |
| (sema3C antisense) | |
| 5′TACACACACACTGCCGATCC-3′; | (SEQ ID NO: 20) |
| (sema3C sense) | |
| 5′CTCACCTGTATGTCTGTGGG-3′. | (SEQ ID NO: 21) |

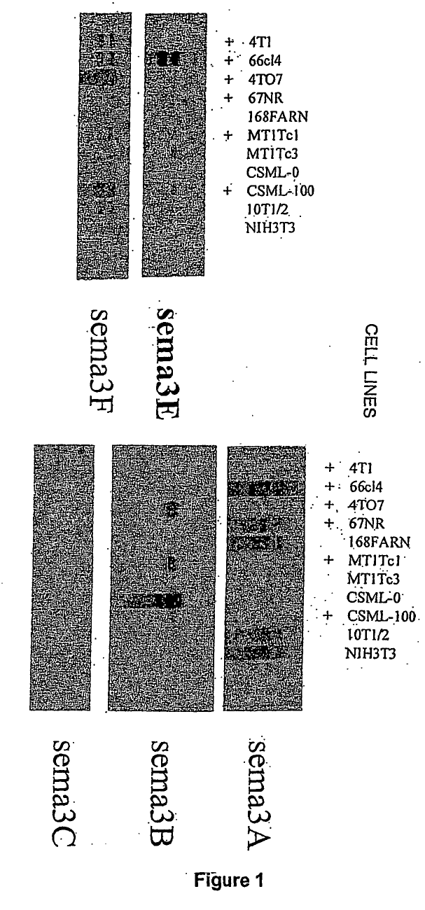

Northern hybridisations were performed as previously described (Christensen et al, 1998, Cancer Res 58:1238-44). FIG. 1 demonstrates Northern hybridisation of subclass 3 semaphorins to RNA from mouse mammary tumor cell lines and immortalised fibroblasts.

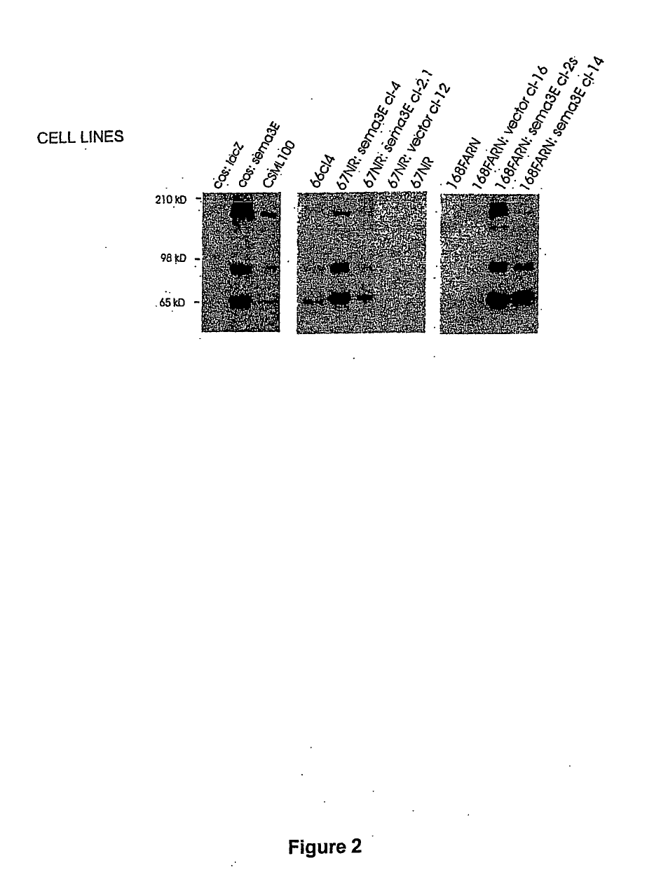

To generate antibodies the sequence encoding the amino terminal part of mouse Sema3E aa 28-98 was released from the pGEX20T plasmid by BamHI restriction and recloned into the pQE30 plasmid, that allowed the production of a histidine tagged-Sema3E fragment in XL1 cells and purification using Ni2+ resin under denaturing conditions using reagents and conditions described in the Xpress protein purification system (Invitrogen). Polyclonal antibodies were raised in rabbits. Boosting was done with sema fragment that had received further purification by electro elution after SDS-PAGE. FIG. 2 shows that these antibodies detect three major isoforms of Sema3E: of about 170 kDa, 90 kDa and 65 kDa.

### Example 2

### Mouse Sema3E and Human SEMA3E Expression Constructs

The pBluescript phagemid containing full-length cDNA of Sema3E was used as the template for the coding part of Sema3E cDNA was amplified by PCR using the sense primer 5′AGAGGAGGGCCCGCCGCCACCATGGCACC-3′ (SEQ ID NO: 22) together with one of two antisense primers, either 5′CGGCAGAGGGGGCCCTCAGGAGAGCAGCG-3′(SEQ ID NO: 23) (encoding a stop codon) or 5′GGGCCCGCGCCCTCGGGAGAGCAGCGTGTG-3′(SEQ ID NO: 24 ) (allowing read through).

The PCR products were cloned into the TOPOTA pCR2.1 vector (Invitrogen). The sema3E sequence was released from the pCR2.1 exploiting Apal sites and recloned into the pcDNA3.1(−)/zeo^{R} to generate a full-length non-tagged Sema3E or into pcDNA3.1 myc-his/neo^{R} to generate a full-length Sema3E containing a myc tag in the carboxy terminal. A pcDNA3.1mychis/neo^{R} encodes a carboxy terminal truncated form of Sema3E lacking the RXXR proteolyic site (sema3E (+)(−)myc). The furin proteolytic site KRRFRR was mutated to KRSFGG by PCR site directed mutagenesis performed on the pCR2.1: Sema3E plasmid using the sense primer

| | |
|---|---|
| 5′-TTCGGCGGGCAGGACGTTCGGCATGGCAACGC | (SEQ ID NO: 25) |
| C-3′ | |
| and | |
| the antisense primer | |
| 5′-GCTTCTCTTTGCGTGTGCACCTGTTGGGTAGT | (SEQ ID NO: 26) |
| A-3′. | |

The PCR was performed using the ExSite PCR site directed Mutagenesis Kit according to the manual (Stratagene). Exploiting the existence of a BamHI site and a EcoRI site in the coding part of the Sema3E cDNA, a cassette containing the mutated site was excised by EcoRI and BamHI digestion, and exchanged with the corresponding wild-type sequence in the pcDNA3.1 /neo^{R}: sema3E(+)(−)myc to obtain the construct encoding sema3E(−)(−)myc, and into the pcDNA3.1/zeo^{R}: sema3E-myc to obtain the sema3E(−)(+)myc. To obtain a construct encoding the secreted p61-Sema3E-myc fused to alkaline phosphotase (AP) in the amino terminal, Sema3E was amplified from the pCR2.1:sema3E using the sense primer 5′CTGCAGAGGCTACGCCTGTCACATAMGMC-3′ (SEQ ID NO: 27) (containing a Pstl linker) and the antisense primer 5′GGGCCCTAGTGCACCTGTTGGGTAGTACCTG-3′ (SEQ ID NO: 28) (containing an Apal linker).

The amplified sequence was cloned into the pSecTag:AP-myc vector to generate the construct encoding AP-p61sema3E-myc. To generate a pcDNA3.1/zeo^{R} construct encoding p61sema3E-myc, pSecTag: AP-p61sema3E-myc was cut with Xhol and Pmel, and the released fragment cloned into the Xhol and Pmel digested pcDNA3.1/zeo^{R}/sema3E-myc.

The Sema3E open reading frame was amplified from the pCR2.1 vector containing the Sema3E PCR fragment (minus stop codon) described above. The sense primer was 5′-GGTCACTCTGCAGGCCCCTCCTACGCCAG-3′ (SEQ ID NO: 29) containing a Pstl site, and the antisense primer was 5′GGGCGGCCGCTCCCTCGGGGG-3′ (SEQ ID NO: 30) containing a Notl site.

A construct encoding the p61 isoform of Sema3E (p61-Sema3E-MH) was engineered from the pPICZαB:sema3E-myc-6×His plasmid. This plasmid was digested with Vnel (site 2066 in the Sema3E sequence submission z80941), followed by treatment with Klenow polymerase, and digestion with Pstl. The released fragment was recloned onto the pPICZαB vector that had received a similar treatment of Xbal digestion, Klenow treatment, and digestion with Pstl.

### Example 3

### Expression of Sema3E-proteins in Pichia Pastoris

The sema3E open reading frame was amplified from the pCR2.1 vector containing the sema3E PCR fragment (minus stop codon) described above. The sense primer was 5′-GGTCACTCTGCAGGCCCCTCCTACGCCAG-3′ (SEQ ID NO: 29) containing a Pstl site, and the antisense primer was 5′GGGCGGCCGCTCCCTCGGGGG-3′(SEQ ID NO: 30) containing a Notl site.

The amplified fragment no longer contained the 5′sequence encoding the native signal peptide of Sema3E. The fragment was cloned into the pPICZαB vector using Pstl and Notl restriction sites giving the Sema3E coding sequence fused in the 5′end to the yeast α-factor sequence under the methanol responsive promoter of the alcohol oxidase gene from Pichia Pastoris (pPICZαB: sema3E-myc-6×His).

A construct encoding the p61 isoform of Sema3E (p61-Sema3E-MH) was engineered from pPICZαB:sema3E-myc-6×His plasmid. This plasmid was digested with Vnel (site 2066 in the sema3E sequence submission z80941), followed by treatment with Klenow polymerase, and digestion with Pstl. The fragment released was recloned onto the pPICZαB vector that had received a similar treatment of Xbal digestion, Klenow treatment, and digestion with Pstl.

The constructs were transformed into the P. Pastois strain GS115 by electroporation, and positive clones were identified by zeocin selection. Experimental conditions were as described in the EasySelect Pichia Expression Kit manual (Invitrogen). Expression of the Sema3E proteins was induced by growing yeast cells in potassium phoshate-buffered minimal medium (pH 6) containing histidin and 0.5% (v/v) methanol. After 72-96 h, expression of sema3E RNA was detected by Northern hybridisation of a ³²P-labelled sema3E probe to yeast RNA isolated using glass beads/phenol-chloroform, whereas detection of Sema3E protein was done by western hybridisation of the growth medium concentrated 50-100 times to anti-Sema3E antibodies, anti-myc antibodies (9E10) or anti-pentahistidine antibodies (Qiagen). The p61-Sema3E-MH protein was purified from the medium using Ni2+ affinity chromatography.

### Example 4

### Stable Transfection of Mammalian Cells with Sema3E

Mouse 168FARN cells were transfected by electroporation. In brief 10⁶ 168FARN cells in 100 μL PBS were mixed with 10 μg plasmid DNA encoding different semahorin constructs and electroporated in a BioRad Gene Pulser at 250 V, 250 μF using Gene Pulser Cuvettes (0.4 cm electrode gap). For establishment of stable transfectants, 4000 cells in 200 μL DMEM+10% FCS were distributed in each well on 96 well plates (Nunc.). The day after the medium was exchanged for DMEM+10% FCS+500 μg/mL zeocin (Invitrogen). Transient transfections of 168FARN cells were made using lipofectamine according to the instructions provided by the manufacturer (Invitrogen).

Monkey kidney COS-7 cells were transfected by the diethylaminoethyl (DEAE) dextran method (Pharmacia-Amersham) or the lipofectamine method (Invitrogen). In the DEAE dextran method, COS-7 cells were grown in 10 cm dishes until they reached ˜80% confluence. They were then washed twice in PBS, and incubated for 30-40 min at 5% CO₂, 37° C. with 2 mL of a 1:1 mixture of 0.5-8 μg plasmid encoding different semahorin constructs diluted in PBS and 0.8 mg/mL of DEAE dextran in PBS. 10 mL DMEM+10% FCS+10 mM chloroquine (Sigma) was added and the cells were left in the incubator for an additional 3 h. The solution was removed and the cells were given a brief shock for 3 min with DMEM+10% FCS+10% DMSO. After washing once in PBS, cells were left to recover over night in DMEM+10% FCS. For collection of conditioned medium, cells were washed with 3×10 mL of serum free DME/F12 and left with 10 mL of this medium for 48-72 h. In the lipofectamine protocol, COS-7 cells were transfected according to the instructions provided by the manufacturer (Invitrogen). A Population of pcDNA3.1/sema3E-transfected cells was maintained in 1000 μg/mL to generate a stable cell line secreting a modest amount of Sema3E.

Human kidney HEK293 cells were transfected using the method of calcium phosphate precipitation. In brief, 4×10⁶ cells in DMEM+10% FBS were seeded in a 10 cm dish the day prior to transfection. 2 h before transfection the growth medium was changed for fresh. 10 μg plasmid were diluted in 450 μL of a 2:1 mixture of 0.1×TE buffer and water, and 50 μL of 2.5 M CaCl₂ was added. To this solution 500 μL of 2×HBS was added drop wise while vortexing (2×HBS: 281 mM NaCl, 100 mM Hepes, 1.5 mM Na₂HPO₄, pH 7.12). The mixture was finally poured onto the HEK293 cells. The CaPᵢ precipitate was allowed to stay on the cells for 16 h, then the cells were prepared for the collection of conditioned medium as described below.

Expression of different Sema proteins is shown in FIG. 2. FIG. 2 demonstrates that Sema3E is expressed by transfected COS-7 and 168FARN cells and can be detected in the medium by immunoblotting.

### Example 5

### Both the Dimer of Full-Length Sema3E and the p61-sema3E Isoform Stimulate Neurite Outgrowth from PC12 Cells

Conditioned medium from COS-7 cells stably transfected with full-length Sema3E was collected and fractionated using heparin-sepharose affinity chromatography and consequent size-exlusion fractionation. Fractions containing different isoforms of Sema3E were collected and tested for neuritogenic activity.

PC12E2 cells were plated on poly-L-lysine coated 48-well plates (Costar) (5,000 cells/well) and grown overnight in DME/F12 medium supplemented with 5% foetal calf serum (FCS) and 10% horse serum (HS) at 37 C°, 5% CO₂. The chromatographic fractions were dialyzed against DME/F12 medium without serum, protein concentration was adjusted to 10 μg/ml, and diluted 1:1 in conditioned medium collected from non-transfected COS-7 cells grown for 72 h in serum free DME/F12 medium. PC12E2 cells were incubated with samples of different Sema3E fractions for 48 h, afterwards cells were fixed with 11% glutaraldehyde in PBS, washed in PBS and stained with 0.1% crystal violet in 20% methanol, and neurite outgrowth was quantified. Quantification was done by counting 100-150 cells/per microscopic field at 125×magnification and then calculating the percentage of cells exhibiting at least one neurite with a length of two cell diameter. A neurite outgrowth index was calculated in each experiment dividing the percentage of neurite outgrowth index in the test sample by the percentage of neurite outgrowth index in response to conditioned medium from COS7 cells without Sema3E proteins (negative control). Other controls were: nerve growth factor (NGF), tested at concentration 500 ng/ml (positive control), and non-fractionated conditioned medium from COS-7 cells expressing full-length Sema3E, tested at concentration 50 μg total protein/mi. Activity was defined as the index divided by 2.

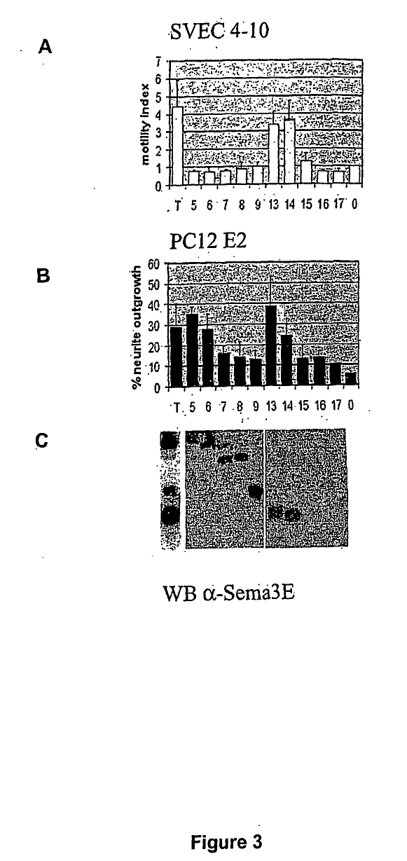

FIG. 3 demonstrates that fractions containing the p87:p87-Sema3E dimer (fractions #5 and #6) and p61-Sema3E (fractions #13 and #14) had the strongest stimulatory effect on neurite outgrowth from PC12E2 cells. The estimated effect was 28-35% for fractions containing the dimer, and 24-39% for the truncated p61-Sema3E isoform, the effect being compared to negative control (conditioned medium from COS-7 cells without Sema3E proteins).

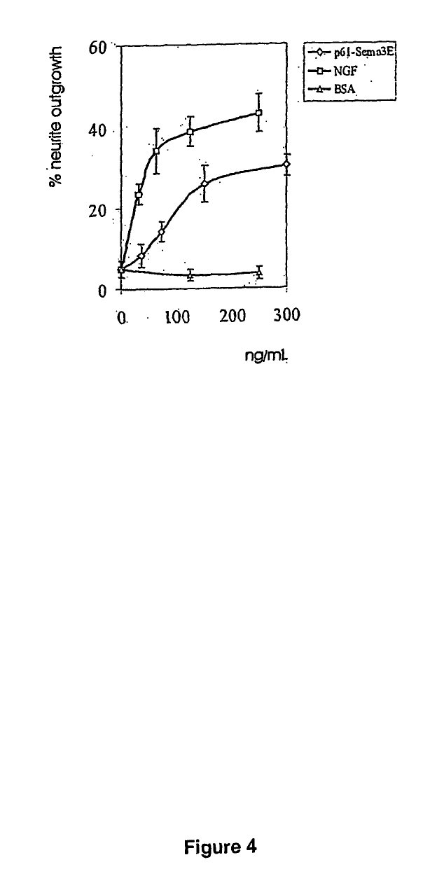

The truncated p61 isoform of Sema3E was further expressed as recombinant protein (p62-Sema3E-MH) in Pichia pastoris as described above, purified using Ni2+ affinity chromatography and tested in a neurite outgrowth assay described above. FIG. 4 shows that p62-Sema3E-MH at concentration 150 ng/ml had a 30% stimulatory effect on neurite outgrowth induction compared to bovine serum albumine (negative control, 125 ng/ml). NGF at (positive control, 125 ng/ml) stimulated neurite outgrowth up to 40%.

### Example 6

### The p61-Sema3E Isoform, but not the Dimer of Full-Length Sema3E Stimulate Endothelial Cell Motility

200.000 endothelial mouse lymph nodes SVEC4-10 cells were suspended either in serum free DME/F12 (1:1) medium containing 25 mM Hepes pH 7.4, 0.1% BSA and D-glucose, or conditioned medium collected from cells expressing different Sema3E constructs and were loaded in the top compartments of blindwell chambers. The cells were left to adhere to fibronectin-coated polycarbonate filters within 3 h after seeding, and allowed to migrate through the pores of the filters in the next 3-6 h. Conditioned media from Sema3E or mock transfected 168FARN cells and COS-7 cells transfected with a pcDNA3.1: LacZ were used to examine the effect of the presence of Sema3E in the medium on endothelial cell motility. Migration of SVEC4-10 cells was evaluated after 5 h exposure to the conditioned media with or without Sema3E.

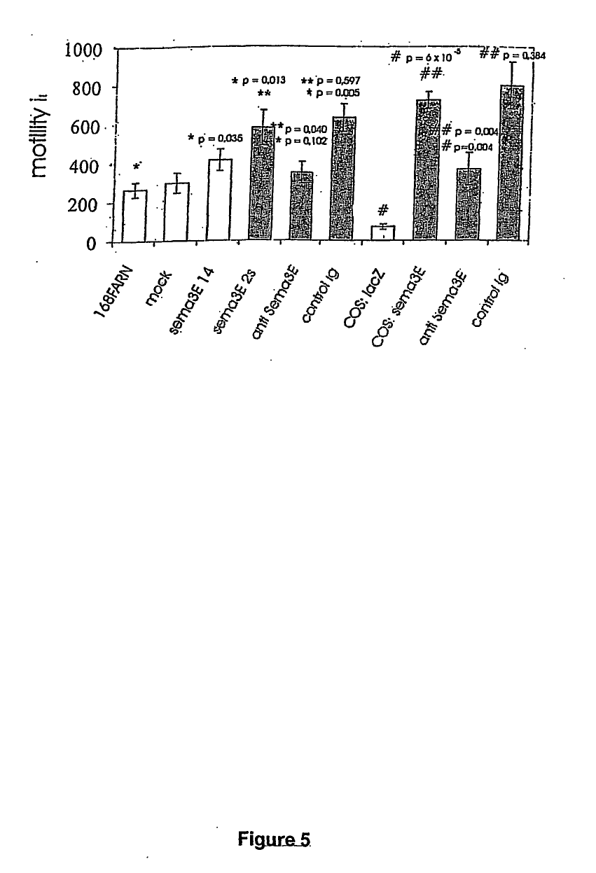

Medium from 168FARN: sema3E clones were ˜2 time more effective in stimulation of cell motility of SVEC4-10 cells than medium from parental 168FARN or mock-transfected cells. Medium from COS-7: sema3E clones were ˜6 times more effective than medium from COS-7: lacZ (FIG. 5).

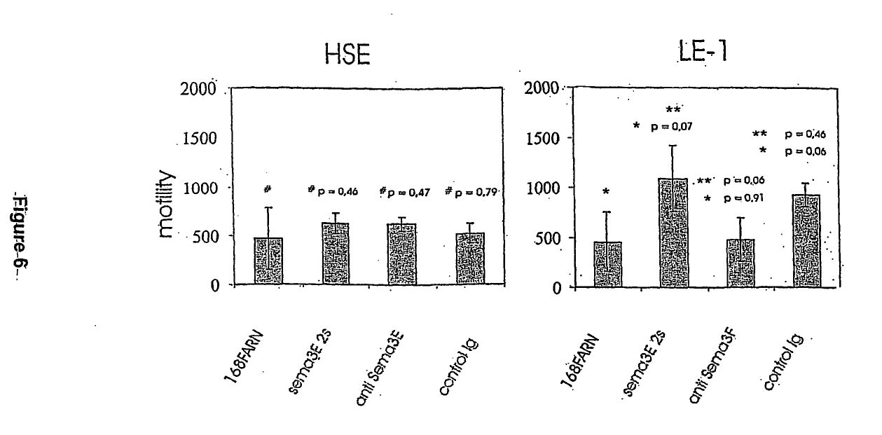

Motility of primary lung microcapillary endothelial cells, LE-1, in response to conditioned medium from 168FARN and 168FARN:sema3E was also increased, whereas motility of hepatic sinusoid endothelial cells, HSE, was not influenced (FIG. 6). This activity was due to the Sema3E protein presence in conditioned medium, since an increase in motility of cells was reduced by immunoprecipitation of Sema3E.

The full-length Sema3E expressed in cells can give a raise to several naturally occurring isoforms of Sema3E, such as for example the truncated p61-Sema3E, a full-length p87-Sema3E, or a dimer p87:p87-Sema3E. The effect of different isoforms of Sema3E on cell motility was studied by using the isoforms purified from conditioned medium collected from tranfected COS-7 cells in the assay described above. The proteins were purified by heparin-sepharose affinity chromatography with following size-exclusion chromatography. All fractions collected from the size-exclusion column were tested for the presence of cell-motility-stimulating activity. FIG. 3 demonstrates that only the p61-Sema3E containing fractions (fractions #13 and #14) were attractive for SVEC4-10 cells.

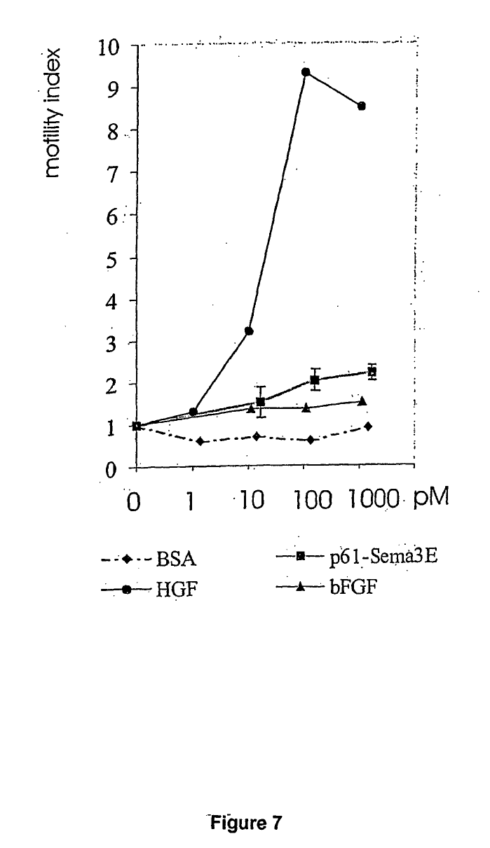

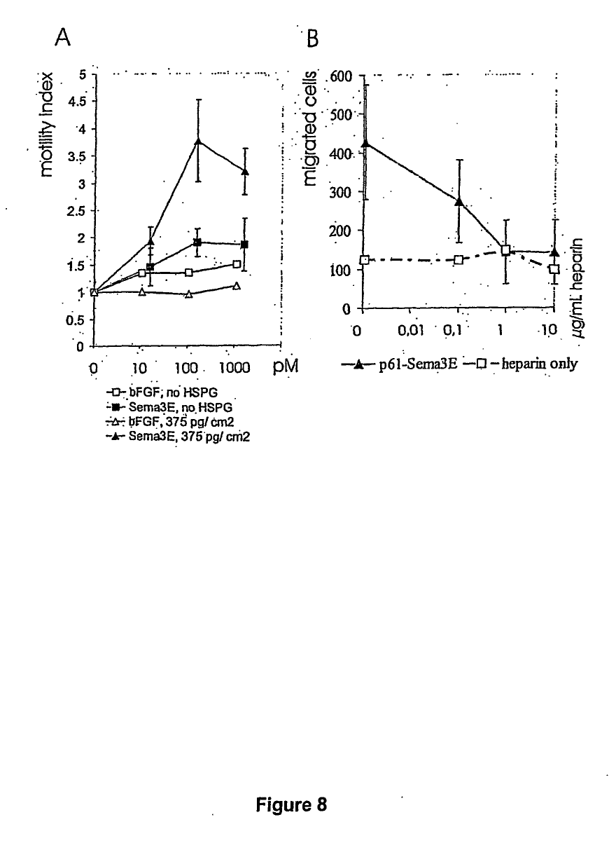

When used 1-100 ng/ml recombinant p62-Sema3E-MH produced in yeast and prepared as described above, the stimulation of cell motility was 2 times higher then in control treatments (FIG. 7). The effect of the recombinant protein was doubled when 375 pg/cm² heparan sulphate proteoglycan (HSPG), Perlican was mixed with fibronectin for coating the adherence filters (FIG. 8A). The presence of soluble heparin in the medium at concentrations 1-10 μg/ml, opposite, decreased the response of SVEC4-10 cells to the p61 -Sema3E-MH to the basal level (FIG. 8B).

### Example 7

### Expression of Sema3E Increases Ability of Tumor Cells to Colonise the Lung in vivo

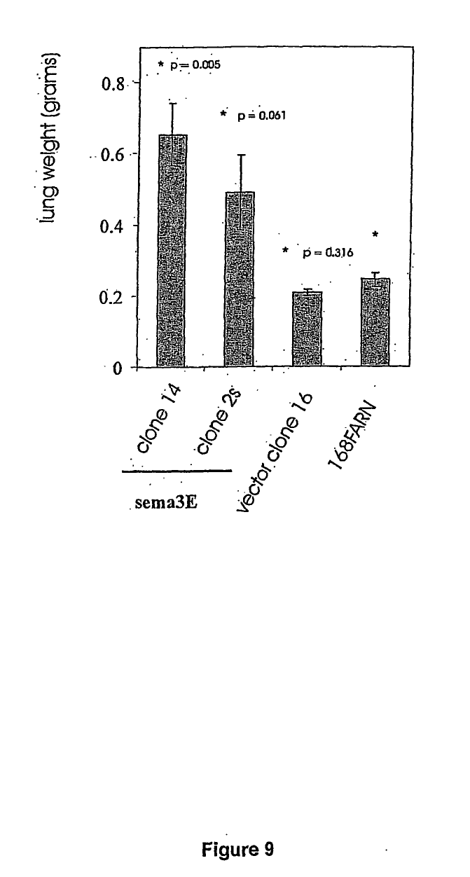

Non-metastatic 168FARN mouse tumor cells were stably transfected with the pcDNA3.1(−)/zeo^{R} vector encoding a wild-type Sema3E, or mock transfected with the empty vector as described above. Two clones expressing secreted Sema3E, 168FARN: 2s and 168FARN: 14, and a mock transfected clone, 168FARN: 16 were tested for their ability to colonise the lungs in the experimental metastasis assay as described previously (Christensen et al, 1998, Cancer Res 58:1238-44). Briefly, 10⁵ cells were injected in the tail vein of female Balb/c mice (age 8-9 weeks). 4 weeks later the lungs were examined for tumor growth. FIG. 9 shows that the growth of Sema3E expressing tumor cells in the lungs increased the overall weight of the lungs by 300%. The sema3E expressing cells, 168FARN: 2s and 168FARN: 14 colonized the lungs and produced large tumors, whereas parental 168FARN cells, or mock transfected cells, 168FARN:16, gave no visible lung tumors.

### Example 8

### Induction the Experimental Lung Metastasis by 168FARN Cells Expressing p61-Sema3E

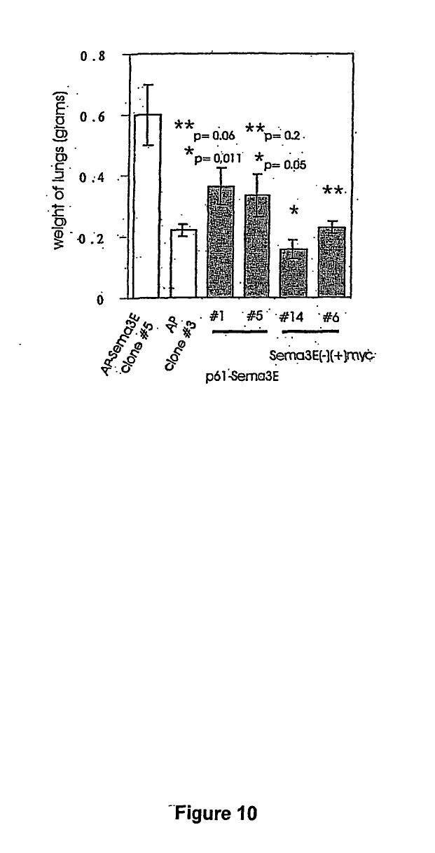

168FARN cells were stably transfected with constructs encoding 1-560 amino acid fragment of the sequence Sema3E, which corresponds to 61 kDa fragment of Sema3E (p61-Sema3E), a full-length Sema3E mutated on KRRFRR site (Sema3E(−) (+)myc), and a wild-type full-length Sema3E as described above, and stable clones expressing the proteins were injected to mice to induce the experimental metastasis. 10⁵ cells of each clone expressing different constructs were injected into the tail vein of female Balb/c mice. After 24 days the lungs were excised, weighted and scored for the presence of visible lung tumors. The p61-Sema3E clones (two groups: clone#1, n=8; clone#5, n=6) were highly aggressive compared to a control group (injected with the cells expressing a secreted alkaline phosphatase (AP)), showing an increase in overall lung weight at about 300%. When these groups were compared to two groups of mice injected with the mutated Sema3E(−)(+)myc (clone#14, n=7; and clone#6, n=7), the difference was about a double increase in the lung weight in the groups with aggressive clones (FIG. 10). The group injected with the cells expressing wild-type sema3E fused with AP (AP-Sema3E) also demonstrated a massive tumor growth, indicating that the 61 kDa fragment of Sema3E is involved in promotion of metastasis independently on the way of delivery of this fragment: either by in vivo proteolysis of full-length Sema3E, or by overexpression.

### Example 9

### 3D Co-Culture of SVEC4-10 and 168FARN

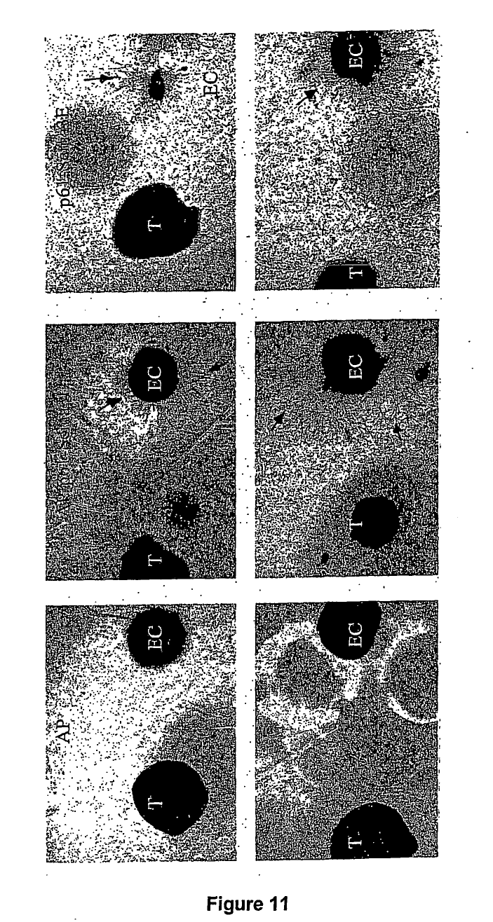

100,000 cells in 10 μl were aggregated by incubation in a hanging drop at 37° C. for 4 h. The aggregates were moved onto a layer of solidified ECM gel (matrigel) so, that SVEC4-10 aggregates were placed 3-5 mm apart from the aggregates of 168FARN:AP (clone#3, control clone expressing AP), or 168FARN:p61-Sema3E (clone#5, expressing the non-tagged p61-sema3E isoform), or 168FARN:AP-p61-Sema3E (done#5, expressing the AP-tagged p61 isoform). Another layer of matrigel was added on the top of the aggregates. After 7 days of incubation aggregates were examined for invasive growth. The invasion of SVEC4-10 endothelial cells was observed in co-cultures with p61-Sema3E transfected 168FARN cells (FIG. 11).

1.-4. (canceled)

5. A method for inhibiting metastasis of cancer in vivo and tumor progression in vitro or in vivo, said method comprising using an agent
i) capable of inhibiting intracellular or extracellular proteolytic processing of a polypeptide belonging to the semaphorin family of proteins,
ii) capable of inhibiting binding of a proteolytic fragment of a polypeptide belonging to the semaphorin family of proteins to a receptor of the Plexin family receptors and thereby inhibiting sequential activation of said receptor.

6. The method according to claim 5, wherein
the cancer is lung, blood, breast, prostate, ovary, brain, kidney, lever, bladder, uterus, haemopoietic tissue, metabolic and endocrine system, epithelia, muscle, bone cancer, or cancer of unknown origin.

7. (canceled)

8. The method according to claim 5, wherein the polypeptide of the semaphorin family belongs to the subclass 3 secreted semaphorins.

9.-10. (canceled)

11. The method according to claim 8, wherein the polypeptide is mouse Sema3E having the amino acid sequence set forth in SEQ ID NO:1.

12. The method according to claim 8, wherein the polypeptide is human SEMA3E having the amino acid sequence set forth in SEQ ID NO:2.

13.-16. (canceled)

17. The method according to claim 5, wherein the agent is a peptide fragment of said semaphorin.

18. The method according to claim 17, wherein the peptide fragment of semaphorin comprises an amino acid sequence RXK/RR.

19.-21. (canceled)

22. The method according to claim 5, wherein the agent is an antibody or a fragment of an antibody, said antibody being raised against said semaphorin, or natural or artificial variants, or peptide fragments thereof, which specifically binds to and inhibits the cleavage of said protein by a serine protease in vivo.

23. The method according to claim 22, wherein the antibody recognises and binds to an epitope located within a sequence of about 5 to about 50 amino acids in length located in the structural domain of the semaphorin comprising a proprotein convertase cleavage site RXK/RR.

24. The method according to claim 22, wherein the antibody is raised against a polypeptide having an amino acid sequence set forth in SEQ ID NO:1 or SEQ ID NO: 2, or fragments thereof.

25.-33. (canceled)

34. (canceled)

35. (canceled)

36. The method according to claim 34, wherein the agent is an antibody as defined in claim 22.

37.-39. (canceled)

40. An antibody, natural or artificial variants thereof, or antibody fragments thereof, which specifically binds to an epitope located within a sequence of about 9 to about 50 amino acids in length located in the structural domain of the semaphorin as defined in claim 8 comprising a proprotein convertase cleavage site RXK/RR, and thereby inhibiting the cleavage of said semaphorin at said cleavage site.

41.-46. (canceled)

47. A method for diagnosis of malignant cancer in an individual having a tumor, comprising
i) assessing the level of expression of the semaphorin as defined in claim 8 in a tumor sample obtained from the individual,
ii) detecting fragments of semaphorin as defined in claim 8 in a body liquid sample, such as blood, urea or faeces, or in a tumor sample obtained from the individual and
iii) measuring the ratio between the full length polypeptide of a semaphorin as defined in claim 8 and peptide fragments of said semaphorin in the tumor and/or a body liquid sample.

48. A method for prognosis of malignancy of cancer in an indibvidual having a cancer, comprising
i) assessing the level of expression of the semaphorin as defined in claim 8 in a tumor sample obtained from said individual, and
ii) detecting fragments of the semaphorin as defined in claim 8 in a body liquid sample, and/or in a tumor sample obtained from said individual and
iii) measuring the ratio between the full length polypeptide of a semaphorin as defined in claim 8 and peptide fragments of said semaphorin in the tumor sample and/or a body liquid sample.

49.-53. (canceled)


SEQUENCE LISTING (30 sequences)

SEQ ID NO: 1 - protein, 775 aa, Mus sp.
MAPAGHILTLLLWGHLLELWTPGHSANPSYPRLRLSHKELLELNRTSIFQSPLGFLDLHTMLLDEYQERLFVGGRDLVYSLNLERVSDGYREIYWPSTAVKVEECIMKGKDANECANYIRVLHHYNRTHLLTCATGAFDPHCAFIRVGHHSEEPLFHLESHRSERGRGRCPFDPNSSFVSTLVGNELFAGLYSDYWGRDSAIFRSMGKLGHIRTEHDDERLLKEPKFVGSYMIPDNEDRDDNKMYFFFTEKALEAENNAHTIYTRVGRLCVNDMGGQRILVNKWSTFLKARLVCSVPGMNGIDTYFDELEDVFLLPTRDPKNPVIFGLFNTTSNIFRGHAVCVYHMSSIREAFNGPYAHKEGPEYHWSLYEGKVPYPRPGSCASKVNGGKYGTTKDYPDDAIRFARIDPLMYQPIKPVHKKPILVKTDGKYNLRQLAVDRVEAEDGQYDVLFIGTDTGIVLKVITIYNQETEWMEEVILEELQIFKDPAPIISMEISSKRQQLYIGSASAVAQVRFHHCDMYGSACADCCLARDPYCAWDGISCSRYYPTGAHAKRRFRRQDVRHGNAAQQCFGQQFVGDALDRTEERLAYGIESNSTLLECTPRSLQAKVIWFYQKGRDVRKEEVKTDDRVVKMDLGLLFLRVRKSDAGTYFCQTVEHNFVHTVRKITLEVVEEHKVEGMFHKDHEEERHHKMPCPPLSGMSQGTKPWYKEFLQLIGYSNFQRVEEYCEKVWCTDKKRKKLKMSPSKWKYANPQEKRLRSKAEHFRLPRHTLLS

SEQ ID NO: 2 - protein, 775 aa, Mus sp.
MASAGHIITLLLWGYLLELWTGGHTADTTHPRLRLSHKELLNLNRTSIFHSPFGFLDLHTMLLDEYQERLFVGGRDLVYSLSLERISDGYKEIHWPSTALKMEECIMKGKDAGECANYVRVLHHYNRTHLLTCGTGAFDPVCAFIRVGYHLEDPLFHLESPRSERGRGRCPFDPSSSFISTLIGSELFAGLYSDYWSRDAAIFRSMGRLAHIRTEHDDERLLKEPKFVGSYMIPDNEDRDDNKVYFFFTEKALEAENNAHAIYTRVGRLCVNDVGGQRILVNKWSTFLKARLVCSVPGMNGIDTYFDELEDVFLLPTRDHKNPVIFGLFNTTSNIFRGHAICVYHMSSIRAAFNGPYAHKEGPEYHWSVYEGKVPYPRPGSCASKVNGGRYGTTKDYPDDAIRFARSHPLMYQAIKPAHKKPILVKTDGKYNLKQIAVDRVEAEDGQYDVLFIGTDNGIVLKVITIYNQEMESMEEVILEELQIFKDPVPIISMEISSKRQQLYIGSASAVAQVRFHHCDMYGSACADCCLARDPYCAWDGISCSRYYPTGTHAKRRFRRQDVRHGNAAQQCFGQQFVGDALDKTEEHLAYGIENNSTLLECTPRSLQAKVIWFVQKGRETRKEEVKTDDRVVKMDLGLLFLRLHKSDAGTYFCQTVEHSFVHTVRKITLEVVEEEKVEDMFNKDDEEDRHHRMPCPAQSSISQGAKPWYKEFLQLIGYSNFQRVEEYCEKVWCTDRKRKKLKMSPSKWKYANPQEKKLRSKPEHYRLPRHTLDS

SEQ ID NO: 3 - protein, 560 aa, Mus sp.
MAPAGHILTLLLWGHLLELWTPGHSANPSYPRLRLSHKELLELNRTSIFQSPLGFLDLHTMLLDEYQERLFVGGRDLVYSLNLERVSDGYREIYWPSTAVKVEECIMKGKDANECANYIRVLHHYNRTHLLTCATGAFDPHCAFIRVGHHSEEPLFHLESHRSERGRGRCPFDPNSSFVSTLVGNELFAGLYSDYWGRDSAIFRSMGKLGHIRTEHDDERLLKEPKFVGSYMIPDNEDRDDNKMYFFFTEKALEAENNAHTIYTRVGRLCVNDMGGQRILVNKWSTFLKARLVCSVPGMNGIDTYFDELEDVFLLPTRDPKNPVIFGLFNTTSNIFRGHAVCVYHMSSIREAFNGPYAHKEGPEYHWSLYEGKVPYPRPGSCASKVNGGKYGTTKDYPDDAIRFARIDPLMYQPIKPVHKKPILVKTDGKYNLRQLAVDRVEAEDGQYDVLFIGTDTGIVLKVITIYNQETEWMEEVILEELQIFKDPAPIISMEISSKRQQLYIGSASAVAQVRFHHCDMYGSACADCCLARDPYCAWDGISCSRYYPTGAHAKRRFRR

SEQ ID NO: 4 - protein, 560 aa, Homo sapiens
MASAGHIITLLLWGYLLELWTGGHTADTTHPRLRLSHKELLNLNRTSIFHSPFGFLDLHTMLLDEYQERLFVGGRDLVYSLSLERISDGYKEIHWPSTALKMEECIMKGKDAGECANYVRVLHHYNRTHLLTCGTGAFDPVCAFIRVGYHLEDPLFHLESPRSERGRGRCPFDPSSSFISTLIGSELFAGLYSDYWSRDAAIFRSMGRLAHIRTEHDDERLLKEPKFVGSYMIPDNEDRDDNKVYFFFTEKALEAENNAHAIYTRVGRLCVNDVGGQRILVNKWSTFLKARLVCSVPGMNGIDTYFDELEDVFLLPTRDHKNPVIFGLFNTTSNIFRGHAICVYHMSSIRAAFNGPYAHKEGPEYHWSVYEGKVPYPRPGSCASKVNGGRYGTTKDYPDDAIRFARSHPLMYQAIKPAHKKPILVKTDGKYNLKQIAVDRVEAEDGQYDVLFIGTDNGIVLKVITIYNQEMESMEEVILEELQIFKDPVPIISMEISSKRQQLYIGSASAVAQVRFHHCDMYGSACADCCLARDPYCAWDGISCSRYYPTGTHAKRRFRR

SEQ ID NO: 5 - protein, 50 aa, Mus sp.
LARDPYCAWDGISCSRYYPTGTHAKRRFRRQDVRHGNAAQQCFGQQFVGD

SEQ ID NO: 6 - protein, 50 aa, Homo sapiens; identical to SEQ ID NO: 5

SEQ ID NO: 7 - DNA, 4460 bp, Mus sp.
aattcggcacgagttcctagaaacgcgcgcgggctcaaccctgcctgaactttcctgtaaacagagcgctgacaggcggcatccccgctgggtggatcccgcgccctggcgcaagtggcacttcttgcttctaattatcgagaggagaggcgaatacgaactagctgctcggcaagtcagtgtcaggaggctgacttctgggaggctggcggggaggctgggggaagagctgggggaggctgctgctctgcttcgactgttttctcaatgaatagctggcggggagactgaagctagccacagcctcctccttcactccgcgtctgggctgacggcgacagcaccagcccggacctggctctcaagacgcgctccttggacggtctcttgctccgcgcttctaaccaccgggcccaaagacagaaaggcttagcggatccaaatattgcccggcaaatggcacttgggaatggtattttctgatgacaaccccttctgtttgtgacaaagcctgtcgcccgccagttgcccctggagggaagtactaagtaaaactcaatcctgtcttaaagtgtggctgcaggggccagaggagagccagcacgcaccatggcaccggccggacacatcctcaccttgctgctctggggtcacctgctggaactctggaccccaggtcactccgcgaacccctcctaccccaggctacgcctgtcacataaagaacttttggaactgaataggacttcaatatttcaaagcccccttggatttcttgatctccatacaatgctgctggatgagtatcaagaacggctctttgtgggaggcagagaccttgtctattccctgaacttggaacgagtcagtgacggctacagagagatatactggccgagcacagcagtaaaggtagaagaatgcataatgaaaggaaaagacgcaaatgagtgtgccaattatatccgggttttgcatcactacaacaggacacaccttctgacctgtgctactggagcttttgatccacactgtgccttcatcagagtcgggcaccattcagaggaacccctgtttcacctggagtcacacagatctgagagaggaaggggcagatgtccttttgaccccaactcctcctttgtgtccacgctagttgggaatgagctgtttgctggactctacagtgactattggggcagagactcggcgatcttccgcagcatggggaagttaggccatattcgcactgagcatgacgatgagcggctcctgaaagaaccaaaatttgtaggttcatatatgattcctgataacgaagaccgagatgacaacaaaatgtactttttctttactgagaaggcgctggaggcggagaacaacgcccacacgatctacacccgagtggggcggctgtgcgtgaatgacatgggaggacagagaatcctggtgaacaagtggagcactttccttaaagcgcggctggtttgctcagtgccgggaatgaatggaatcgacacatactttgacgaactagaggatgtgtttttactgccgaccagagatcctaagaatccagtgatatttggactgtttaatactaccagcaatatatttagaggccatgctgtatgtgtgtatcacatgtcaagtatccgggaagcctttaatggcccatatgctcataaagaaggccctgaataccactggtcactatatgaaggaaaagtcccctacccaaggcctggttcctgtgccagcaaagtaaacggaggcaagtatggaaccaccaaagattaccccgatgacgccatccggttcgcaaggatcgatcctctaatgtatcagcccataaaacctgttcataaaaaaccaatactggtaaaaacagatggaaaatacaacctgaggcaacttgccgtggatcgggtggaagcggaggatggccagtatgacgtcttatttattgggacagacacaggaattgtgctgaaagtaatcacaatttacaaccaagaaacagagtggatggaggaagtcattctagaggaacttcaaatattcaaggatccagcccctatcatttctatggaaatttcttcaaagagacaacagctttacattggatcagcctctgctgtggcacaagtcagattccatcactgcgacatgtatggcagtgcttgtgctgactgctgcctggctcgagacccgtactgtgcctgggatggcatatcctgctccaggtactacccaacaggtgcacacgcaaagaggaggttccgcaggcaggacgttcggcatggcaacgccgcccaacagtgctttggacagcaatttgttggagacgcgttggacaggactgaagagaggctggcttatggcatagagagcaacagtactctgttggaatgcaccccgcgatcactacaagcaaaagtcatctggtttgtacagaagggacgcgacgtaagaaaagaagaggtgaagacggatgacagagttgtcaagatggacttgggcttgctcttcctcagagtacgcaagtcagatgcagggacctatttttgccagacagtagaacacaattttgtccatactgtgcgtaaaatcaccttggaggtggtcgaagagcataaagtggagggcatgtttcataaggaccatgaagaggaaagacatcacaagatgccctgccctcccttaagcggtatgtctcaggggacaaaaccgtggtacaaggaattcttgcagctgattggctacagcaacttccagagagtggaagaatactgcgaaaaggtgtggtgtacagataagaagaggaaaaagcttaaaatgtctccctccaagtggaagtatgccaacccccaggaaaagaggcttcgctctaaagctgagcacttccgcctgcccaggcacacgctgctctcctgagggcgccctctgccggcggctgaggaacctaggatggaaacatttttttaaagggggggaaaaaaaaaagactgaaagcatgcagtctctttcattacttcaaagaactttctgtagtactgagaggctgggaaggtgttttaagttattctgcatatcatctgactgtacaaacatctctctcacagtactagtacttaaacgagtgtattgctttaaaaggcttcacctatttatcaacatatgatcgggctgtaattttgagcttgcttttcagggaaattattttgccattgggatatgtattataattataatattaaagaaaaagttataggttttcataatcaagaaaaatgtaaaactctttctggagttttttcttttttcttttcctctttgcagccataaggtaaaatatacagaatatcagatattgtagaattatcgtgtgagctttctggtcagttgttgatttttttttttaagccataagttgtatgtagctgtgttgtggttgctataaactgtacaagaaatctgcaacccatccattctgaggcttgttctcttcatgaaacactcactaaactatattaagaatatttctgttttataaacaagacatatcttttaaatttcacttcagtgtggaaggatggaagatagcctctcagaaagatatttttaaatagtttcaaaccatgaaagtagaattctgttaaaaacggtctgtgcttccctcttgtggtacaagaaggcattcgatctctcaatgctgtttccataattttcctcgaatgacttgaaaacaaaatgtagtagatacatctagttcactattcagaagactcaattaaaaaataattttatataataatttaatatggaaatagcatgcttctggttaattcattacttcacaacaaaccgaagatgttaactgcatttgagctttgaaatagaagaaagtacctttccagtcctaccctgagacaagacactctgaaagtgtgctttacccgcaaagaaattatcaacgattcttttctcagaaatgaaatcttcctattttatatttacttcttggattcgttttactactgacatctttagagaatcattatctttgtcttatgaatataatcaaaatttatatttttcagaatgtttatttcttgagatttttcttctctgtattttaaagagccagcagcttccgttctaaaatatgatttttatgaggcttttcagttcggtaagagtaacgcaagtggattgtaataagaacaattatgaatacgtattcagacttaaatttaactattatgtcagctatctcagtatgctatataattaagaatataaagtgttgagaacttctaatggatttctttttctgtctaaaattaggtactacataaatattccacaattttcaaattttaaaattttggaaattttaaagtccagaatttctaaaacatataactagtatgaataaagagaatgttgacatcctc

SEQ ID NO: 8 - DNA, 2328 bp, Mus sp.
atggcaccggccggacacatcctcaccttgctgctctggggtcacctgctggaactctggaccccaggtcactccgcgaacccctcctaccccaggctacgcctgtcacataaagaacttttggaactgaataggacttcaatatttcaaagcccccttggatttcttgatctccatacaatgctgctggatgagtatcaagaacggctctttgtgggaggcagagaccttgtctattccctgaacttggaacgagtcagtgacggctacagagagatatactggccgagcacagcagtaaaggtagaagaatgcataatgaaaggaaaagacgcaaatgagtgtgccaattatatccgggttttgcatcactacaacaggacacaccttctgacctgtgctactggagcttttgatccacactgtgccttcatcagagtcgggcaccattcagaggaacccctgtttcacctggagtcacacagatctgagagaggaaggggcagatgtccttttgaccccaactcctcctttgtgtccacgctagttgggaatgagctgtttgctggactctacagtgactattggggcagagactcggcgatcttccgcagcatggggaagttaggccatattcgcactgagcatgacgatgagcggctcctgaaagaaccaaaatttgtaggttcatatatgattcctgataacgaagaccgagatgacaacaaaatgtactttttctttactgagaaggcgctggaggcggagaacaacgcccacacgatctacacccgagtggggcggctgtgcgtgaatgacatgggaggacagagaatcctggtgaacaagtggagcactttccttaaagcgcggctggtttgctcagtgccgggaatgaatggaatcgacacatactttgacgaactagaggatgtgtttttactgccgaccagagatcctaagaatccagtgatatttggactgtttaatactaccagcaatatatttagaggccatgctgtatgtgtgtatcacatgtcaagtatccgggaagcctttaatggcccatatgctcataaagaaggccctgaataccactggtcactatatgaaggaaaagtcccctacccaaggcctggttcctgtgccagcaaagtaaacggaggcaagtatggaaccaccaaagattaccccgatgacgccatccggttcgcaaggatcgatcctctaatgtatcagcccataaaacctgttcataaaaaaccaatactggtaaaaacagatggaaaatacaacctgaggcaacttgccgtggatcgggtggaagcggaggatggccagtatgacgtcttatttattgggacagacacaggaattgtgctgaaagtaatcacaatttacaaccaagaaacagagtggatggaggaagtcattctagaggaacttcaaatattcaaggatccagcccctatcatttctatggaaatttcttcaaagagacaacagctttacattggatcagcctctgctgtggcacaagtcagattccatcactgcgacatgtatggcagtgcttgtgctgactgctgcctggctcgagacccgtactgtgcctgggatggcatatcctgctccaggtactacccaacaggtgcacacgcaaagaggaggttccgcaggcaggacgttcggcatggcaacgccgcccaacagtgctttggacagcaatttgttggagacgcgttggacaggactgaagagaggctggcttatggcatagagagcaacagtactctgttggaatgcaccccgcgatcactacaagcaaaagtcatctggtttgtacagaagggacgcgacgtaagaaaagaagaggtgaagacggatgacagagttgtcaagatggacttgggcttgctcttcctcagagtacgcaagtcagatgcagggacctatttttgccagacagtagaacacaattttgtccatactgtgcgtaaaatcaccttggaggtggtcgaagagcataaagtggagggcatgtttcataaggaccatgaagaggaaagacatcacaagatgccctgccctcccttaagcggtatgtctcaggggacaaaaccgtggtacaaggaattcttgcagctgattggctacagcaacttccagagagtggaagaatactgcgaaaaggtgtggtgtacagataagaagaggaaaaagcttaaaatgtctccctccaagtggaagtatgccaacccccaggaaaagaggcttcgctctaaagctgagcacttccgcctgcccaggcacacgctgctctcctga

SEQ ID NO: 9 - DNA, 3982 bp, Mus sp.
aattcggcacgagttcctagaaacgcgcgcgggctcaaccctgcctgaactttcctgtaaacagagcgctgacaggcggcatccccgctgggtggatcccgcgccctggcgcaagtggcacttcttgcttctaattatcgagaggagaggcgaatacgaactagctgctcggcaagtcagtgtcaggaggctgacttctgggaggctggcggggaggctgggggaagagctgggggaggctgctgctctgcttcgactgttttctcaatgaatagctggcggggagactgaagctagccacagcctcctccttcactccgcgtctgggctgacggcgacagcaccagcccggacctggctctcaagacgcgctccttggacggtctcttgctccgcgcttctaaccaccgggcccaaagacagaaaggcttagcggatccaaatattgcccggcaaatggcacttgggaatggtattttctgatgacaaccccttctgtttgtgacaaagcctgtcgcccgccagttgcccctggagggaagtactaagtaaaactcaatcctgtcttaaagtgtggctgcaggggccagaggagagccagcacgcaccatggcaccggccggacacatcctcaccttgctgctctggggtcacctgctggaactctggaccccaggtcactccgcgaacccctcctaccccaggctacgcctgtcacataaagaacttttggaactgaataggacttcaatatttcaaagcccccttggatttcttgatctccatacaatgctgctggatgagtatcaagaacggctctttgtgggaggcagagaccttgtctattccctgaacttggaacgagtcagtgacggctacagagagatatactggccgagcacagcagtaaaggtagaagaatgcataatgaaaggaaaagacgcaaatgagtgtgccaattatatccgggttttgcatcactacaacaggacacaccttctgacctgtgctactggagcttttgatccacactgtgccttcatcagagtcgggcaccattcagaggaacccctgtttcacctggagtcacacagatctgagagaggaaggggcagatgtccttttgaccccaactcctcctttgtgtccacgctagttgggaatgagctgtttgctggactctacagtgactattggggcagagactcggcgatcttccgcagcatggggaagttaggccatattcgcactgagcatgacgatgagcggctcctgaaagaaccaaaatttgtaggttcatatatgattcctgataacgaagaccgagatgacaacaaaatgtactttttctttactgagaaggcgctggaggcggagaacaacgcccacacgatctacacccgagtggggcggctgtgcgtgaatgacatgggaggacagagaatcctggtgaacaagtggagcactttccttaaagcgcggctggtttgctcagtgccgggaatgaatggaatcgacacatactttgacgaactagaggatgtgtttttactgccgaccagagatcctaagaatccagtgatatttggactgtttaatactaccagcaatatatttagaggccatgctgtatgtgtgtatcacatgtcaagtatccgggaagcctttaatggcccatatgctcataaagaaggccctgaataccactggtcactatatgaaggaaaagtcccctacccaaggcctggttcctgtgccagcaaagtaaacggaggcaagtatggaaccaccaaagattaccccgatgacgccatccggttcgcaaggatcgatcctctaatgtatcagcccataaaacctgttcataaaaaaccaatactggtaaaaacagatggaaaatacaacctgaggcaacttgccgtggatcgggtggaagcggaggatggccagtatgacgtcttatttattgggacagacacaggaattgtgctgaaagtaatcacaatttacaaccaagaaacagagtggatggaggaagtcattctagaggaacttcaaatattcaaggatccagcccctatcatttctatggaaatttcttcaaagagacaacagctttacattggatcagcctctgctgtggcacaagtcagattccatcactgcgacatgtatggcagtgcttgtgctgactgctgcctggctcgagacccgtactgtgcctgggatggcatatcctgctccaggtactacccaacaggtgcacacgcaaagaggaggttccgcaggcaggacgttcggcatggcaacgccgcccaacagtgctttggacagcaatttgttggagacgcgttggacaggactgaagagaggctggcttatggcatagagagcaacagtactctgttggaatgcaccccgcgatcactacaagcaaaagtcatctggtttgtacagaagggacgcgacgtaagaaaagaagaggtgaagacggatgacagagttgtcaagatggacttgggcttgctcttcctcagagtacgcaagtcagatgcagggacctatttttgccagacagtagaacacaattttgtccatactgtgcgtaaaatcaccttggaggtggtcgaagagcataaagtggagggcatgtttcataaggaccatgaagaggaaagacatcacaagatgccctgccctcccttaagcggtatgtctcaggggacaaaaccgtggtacaaggaattcttgcagctgattggctacagcaacttccagagagtggaagaatactgcgaaaaggtgtggtgtacagataagaagaggaaaaagcttaaaatgtctccctccaagtggaagtatgccaacccccaggaaaagaggcttcgctctaaagctgagcacttccgcctgcccaggcacacgctgctctcctgagggcgccctctgccggcggctgaggaacctaggatggaaacatttttttaaagggggggaaaaaaaaaagactgaaagcatgcagtctctttcattacttcaaagaactttctgtagtactgagaggctgggaaggtgttttaagttattctgcatatcatctgactgtacaaacatctctctcacagtactagtacttaaacgagtgtattgctttaaaaggcttcacctatttatcaacatatgatcgggctgtaattttgagcttgcttttcagggaaattattttgccattgggatatgtattataattataatattaaagaaaaagttataggttttcataatcaagaaaaatgtaaaactctttctggagttttttcttttttcttttcctctttgcagccataaggtaaaatatacagaatatcagatattgtagaattatcgtgtgagctttctggtcagttgttgatttttttttttaagccataagttgtatgtagctgtgttgtggttgctataaactgtacaagaaatctgcaacccatccattctgaggcttgttctcttcatgaaacactcactaaactatattaagaatatttctgttttataaacaagacatatcttttaaatttcacttcagtgtggaaggatggaagatagcctctcagaaagatatttttaaatagtttcaaaccatgaaagtagaattctgttaaaaacggtctgtgcttccctcttgtggtacaagaaggcattcgatctctcaatgctgtttccataattttcctcgaatgacttgaaaacaaaatgtagtagatacatctagttcactattcagaagactcaattaaaaaataatttttatataattaagaatataaagtgttgagaacttctaatggatttctttttctgtctaaaattaggtactacataaatattccacaattttcaaattttaaaattttggaaattttaaagtccagaatttctaaaacatataactagtatgaataaagagaatgttgacatcctc

SEQ ID NO: 10 - DNA, 1200 bp, Homo sapiens
gtttggcaagtcagtgcaagaggctgacttctgagaggcttccaggagcccgaagagaggacctccacgggagaagggagtgcgtgtgctcggttttttttttttctctcttttttttttttttttctgaatgaacagctttgcccaagtgactgaaaaatacagcttcttcctgaatctaccggcgtagttgctgaagagcgctctagacaggacatggctctgaagactcactctttggaatgtcctcttgctcccggcttataaacaactgtcccgaggaaagaaaggttttacatagccaaatacagcctgacaaatggcacttcggaactgtgctttctgatgacaacgcgttcgatttctgacaaagcctctcgcacgctgcccctggagggaagtcctaagtaaaactcagaccctccttaaagtgaggagcgagggcttggacggtgaacacggcagcatggcatccgcggggcacattatcaccttgctcctgtggggttacttactggagctttggacaggaggtcatacagctgatactacccacccccggttacgcctgtcacataaagagctcttgaatctgaacagaacatcaatatttcatagcccttttggatttcttgatctccatacaatgctgctggatgaatatcaagagaggctcttcgtgggaggcagggaccttgtatattccctcagcttggagagaatcagtgacggctataaagagatacactggccgagtacagctctaaaaatggaagaatgcataatgaagggaaaagatgcgggtgaatgtgcaaattatgttcgggttttgcatcactataacaggacacaccttctgacctgtggtactggagcttttgatccagtttgtgccttcatcagagttggatatcatttggaggatcctctgtttcacctggaatcacccagatctgagagaggaaggggcagatgtccttttgaccccagctcctccttcatctccactttaattggtagtgaattgtttgctggactctacagtgactactggagcagagacgctgcgatcttccgcagcatggggcgactggcccatatccgcactgagcatgacgatgagcgtctgttgaaagaaccaaaatttgtaggttcatacatgattcctgacaatgaagacagagatgacaacaaagtata

SEQ ID NO: 11 - protein, 680 aa, Mus sp.
CVQLTVQPRNVSVTMSQVPLVLQAWNVPDLSAGVNCSFEDFTESESVLEDGRIHCRSPSAREVAPITRGQGDQRVVKLYGKSKETGKKFASVDFVFYNCSVHQSCLSCVNGSFPCHWCKYRHVCTHNVADCAFLEGRVNVSEDCPQILPSTQIYVPVGVVKPITLAARNLPQPQSGQRGYECLFHIPGSPARVTALRFNSSSLQCQNSSYSYEGNDVSDLPVNLSVVWNGNFVIDNPQNIQAHLYKCPALRESCGLCLKADPRFECGWCVAERRCSLRHHCAADTPASWMHARHGSSRCTDPKILKLSPETGPRQGGTRLTITGENLGLRFEDVRLGVRVGKVLCSPVESEYISAEQIVCEIGDASSVRAHDALVEVCVRDCSPHYRALSPKRFTFVTPTFYRVSPSRGPLSGGTWIGIEGSHLNAGSDVAVSVGGRPCSFSWRNSREIRCLTPPGQSPGSAPIIININRAQLTNPEVKYNYTEDPTILRIDPEWSINSGGTLLTVTGTNLATVREPRIRAKYGGIERENGCLVYNDTTMVCRAPSVANPVRSPPELGERPDELGFVMDNVRSLLVLNSTSFLYYPDPVLEPLSPTGLLELKPSSPLILKGRNLLPPAPGNSRLNYTVLIGSTPCTLTVSETQLLCEAPNLTGQHKVTVRAGGFEFSPGTLQVYSDSLLT

SEQ ID NO: 12 - protein, 677 aa, Mus sp.
CVSLAVHPSSISVSEHSRLLSLVVSDAPDLSAGIACAFGNLTEVEGQVSGSQVICISPGPKDVPVIPLDQDWFGLELQLRSKETGKIFVSTEFKFYNCSAHQLCLSCVNSAFRCHWCKYRNLCTHDPTTCSFQEGRINISEDCPQLVPTEEILIPVGEVKPITLKARNLPQPQSGQRGYECVLNIQGAIHRVPALRFNSSSVQCQNSSYQYDGMDISNLAVDFAVVWNGNFIIDNPQDLKVHLYKCAAQRESCGLCLKADRKFECGWCSGERRCTLHQHCTSPSSPWLDWSSHNVKCSNPQITEILTVSGPPEGGTRVTIHGVNLGLDFSEIAHHVQVAGVPCTPLPGEYIIAEQIVCEMGHALVGTTSGPVRLCIGECKPEFMTKSHQQYTFVNPSVLSLNPIRGPESGGTMVTITGHYLGAGSSVAVYLGNQTCEFYGRSMSEIVCVSPPSSNGLGPVPVSVSVDRAHVDSNLQFEYIDDPRVQRIEPEWSIASGHTPLTITGFNLDVIQEPRIRVKFNGKESVNVCKVVNTTTLTCLAPSLTTDYRPGLDTVERPDEFGFVFNNVQSLLIYNDTKFIYYPNPTFELLSPTGVLDQKPGSPIILKGKNLCPPASGGAKLNYTVLIGETPCAVTVSETQLLCEPPNLTGQHKVMVHVGGMVFSPGSVSVISDSLLT

SEQ ID NO: 13 - protein, 687 aa, Mus sp.
GFAEELSKCVQVRVRPNNVSVTSPGVQLTVTLHNVPDLSAGVSCAFEAAAENEAVLLPSGELLCPSPSLQELRALTRGHGATRTVRLQLLSKETGVRFAGADFVFYNCSVLQSCMSCVGSPYPCHWCKYRHTCTSRPHECSFQEGRVHSPEGCPEILPSGDLLIPVGVMQPLTLRAKNLPQPQSGQKNYECVVRVQGRQQRVPAVRFNSSSVQCQNASYSYEGDEHGDTELDFSVVWDGDFPIDKPPSFRALLYKCWAQRPSCGLCLKADPRFNCGWCISEHRCQLRTHCPAPKTNWMHLSQKGTRCSHPRITQIHPLVGPKEGGTRVTIVGENLGLLSREVGLRVAGVRCNSIPAEYISAERIVCEMEESLVPSPPPGPVELCVGDCSADFRTQSEQVYSFVTPTFDQVSPSRGPASGGTRLTISGSSLDAGSRVTVTVRDSECQFVRRDAKAIVCISPLSTLGPSQAPITLAIDRANISSPGLIYTYTQDPTVTRLEPTWSIINGSTAITVSGTHLLTVQEPRVRAKYRGIETTNTCQVINDTAMLCKAPGIFLGRPQPRAQGEHPDEFGFLLDHVQTARSLNRSSFTYYPDPSFEPLGPSGVLDVKPGSHVVLKGKNLIPAAAGSSRLNYTVLIGGQPCSLTVSDTQLLCDSPSQTGRQPVMVLVGGLEFWLGTLHISAERALT

SEQ ID NO: 14 - DNA, 22 bp, Artificial sequence
  SEMA 3E sense primer
cgggatccatttttttacgaaa

SEQ ID NO: 15 - DNA, 23 bp, Artificial sequence
  SEMA 3E antisense primer
gcggatcctcccagccataggtc

SEQ ID NO: 16 - DNA, 20 bp, Artificial sequence
  sema 3E antisense primer
acatgcacacagcagatccc

SEQ ID NO: 17 - DNA, 20 bp, Artificial sequence
  sema 3E sense primer
ggaagagcccttatgatccc

SEQ ID NO: 18 - DNA, 20 bp, Artificial sequence
  sema 3B antisense primer
caactccaggtactgagcac

SEQ ID NO: 19 - DNA, 20 bp, Artificial sequence
  sema 3B sense primer
aatgcaactgggcagggaag

SEQ ID NO: 20 - DNA, 20 bp, Artificial sequence
  sema 3C antisense primer
tacacacacactgccgatcc

SEQ ID NO: 21 - DNA, 20 bp, Artificial sequence
  sema 3C sense primer
ctcacctgtatgtctgtggg

SEQ ID NO: 22 - DNA, 29 bp, Artificial sequence
  sema 3E sense primer
agaggagggcccgccgccaccatggcacc

SEQ ID NO: 23 - DNA, 29 bp, Artificial sequence
  sema 3E antisense primer including stop codon
cggcagagggggccctcaggagagcagcg

SEQ ID NO: 24 - DNA, 30 bp, Artificial sequence
  sema 3E antisense primer (read through)
gggcccgcgccctcgggagagcagcgtgtg

SEQ ID NO: 25 - DNA, 33 bp, Artificial sequence
  sema 3E sense primer (mutated proteolitic site)
ttcggcgggcaggacgttcggcatggcaacgcc

SEQ ID NO: 26 - DNA, 33 bp, Artificial sequence
  sema 3E antisense primer (mutated proteolitic site)
gcttctctttgcgtgtgcacctgttgggtagta

SEQ ID NO: 27 - DNA, 31 bp, Artificial sequence
  sema 3E sense primer (Pst I linker)
ctgcagaggctacgcctgtcacataaagaac

SEQ ID NO: 28 - DNA, 31 bp, Artificial sequence
  sema 3E antisense primer (Apa I linker)
gggccctagtgcacctgttgggtagtacctg

SEQ ID NO: 29 - DNA, 29 bp, Artificial sequence
  sema 3E sense primer (Pst I site)
ggtcactctgcaggcccctcctacgccag

SEQ ID NO: 30 - DNA, 21 bp, Artificial sequence
  sema 3E antisense primer (Not I site)
gggcggccgctccctcggggg